SEQUENCE LISTING (4 sequences)

SEQ ID NO: 1 - DNA, 1387 bp, Bacillus methylotrophicus
  source 1..1387: mol_type = genomic DNA
gttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctattcgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcaaacaaccatccggtattagccccggtttcccggagttatcccagtcttacaggcaggttacccacgtgttactcacccgtccgccgctaacatcagggagcaagctcccatctgtc

SEQ ID NO: 2 - DNA, 1274 bp, Bacillus subtilis
  source 1..1274: mol_type = genomic DNA
ggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtaccgccctattcgaacggtacttgttcttccctaacaacagagctttacgatccgaaaaccttcatcactcacgcggcgttgctccgtcagactttcgtccattgcggaagattccctactgctgcctcccgtaggagtctgggscgtgtctcagtcccagtgtggccgatcaccctctcaggtcggctacgcatcgttgccttggtgagccattacctcaccaactagctaatgcgccgcgggtccatctgtaagtggtagccgaagccaccttttatgtttgaaccatgcggttcaaacaaccatccggtattagccccggtttcccggagttat

SEQ ID NO: 3 - DNA, 1379 bp, Bacillus amyloliquefaciens
  source 1..1379: mol_type = genomic DNA
ggttacctcaccgacttcgggtgttacaaactctcgtggtgtgacgggcggtgtgtacaaggcccgggaacgtattcaccgcggcatgctagatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcaaacaaccatccggtattagccccggtttcccggagttatcccagtcttacaggcaggttacccacgtgttactcacccgtccgccgctaacatcagggagcaagct

SEQ ID NO: 4 - DNA, 1361 bp, Bacillus amyloliquefaciens
  source 1..1361: mol_type = genomic DNA
acctcaccgacttcgggtgttacaaactctcgtggtgtgacgggcggtgtgtacaaggcccgggaacgtattcaccgcggcatgctrgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaaca

(19) United States
(12) Patent Application Publication
(10) Pub. No.: US 2026/0337549 A1
(43) Pub. Date: Oct. 29, 2026

(54) ODOR CONTROLLING BACTERIA

(71) Applicant: BASF SE, Ludwigshafen Am Rhein (DE)

(72) Inventors: Christoph SCHILLING, Ludwigshafen am Rhein (DE); Andrea HEROLD, Ludwigshafen am Rhein (DE); Oliver SPANGENBERG, Ludwigshafen am Rhein (DE); Menno HAZENKAMP, Grenzach-Wyhlen (DE); Dietmar OCHS, Grenzach-Wyhlen (DE); Barbara NAVE, Ludwigshafen am Rhein (DE)

(73) Assignee: BASF SE, Ludwigshafen Am Rhein (DE)

(21) Appl. No.: 19/122,342

(22) PCT Filed: Oct. 11, 2023
(86) PCT No.: PCT/EP2023/078168

§ 371 Date: Apr. 18, 2025

(30) Foreign Application Priority Data
Oct. 20, 2022 (EP) 22202842.5
Apr. 6, 2023 (EP) 23167096.9

(51) Int. Cl. C12N 1/205; A01N 63/22; A01P 1/00; C11D 3/38; C12R 1/07; C12R 1/125

(52) U.S. Cl. CPC C12N 1/205; A01N 63/22; A01P 1/00; C11D 3/381; C12R 2001/07; C12R 2001/125

(57) ABSTRACT

In the present invention new Bacillus strains are provided. More specifically, Bacillus strains having extracellular hydrolase activity and demonstrating reduced malodor after incubation for at least 5 days for at least 25 °C in a lactose-free whole milk sample compared to a non-inoculated lactose-free whole milk sample, a composition comprising these strains and methods using the new Bacillus strains are provided.

### FIELD OF THE INVENTION

In the present invention new Bacillus strains are provided. More specifically, Bacillus strains having extracellular hydrolase activity and demonstrating reduced malodor after incubation for at least 5 days for at least 25 °C in a lactose-free whole milk sample compared to a non-inoculated lactose-free whole milk sample, a composition comprising these strains and methods using the new Bacillus strains are provided.

### BACKGROUND

Microorganisms generally live attached to surfaces in many natural, industrial, and medical environments, encapsulated by extracellular substances including biopolymers and macromolecules. Exposed to suitable conditions these microorganisms can grow and effect malodor caused by their own metabolites or by forming biofilms that provide a sticky area for malodor molecules of the surroundings.

For example, when laundry items are used, they are contacted to sweat and microorganisms from the body of the user. Some of these microorganisms, especially bacteria, are capable of adhering to the laundry item and form a biofilm on the item. Due to the above mentioned reasons, the presence of the microorganisms causes the laundry items to smell.

Also, food or dirty household items, such as tiles, dishes, cups and cutlery, comprising residues of food or beverage may demonstrate increasing malodor based on microbial growth over the course of time. For instance, milk is an excellent growth medium for microorganisms, in particular bacteria. It provides the nutrients and moisture and has a near neutral pH. Off-flavors and/or malodors in milk are the results of bacterial growth, named psychrotrophs. Psychrotrophs include many kinds of bacteria, all of which cause milk spoilage. Their ideal growth temperature is 18-22 °C. Cold temperatures slow their growth, but do not kill them. At 7 °C or above, their growth is steady and off-flavors and/or malodors can be present within two to three days.

Malty flavor in milk tastes like Grape-Nuts cereal. The cause is the presence and growth of Streptococcus lactis in poorly cooled milk. Said flavor is generally a forerunner of high acid flavors and malodors. They are rarely developed in pasteurized milk. However, if not stopped by pasteurization, a malty flavor will later become a high acidic one.

Bacteria and biofilms may also occur on surfaces of sanitary equipment, such as toilets, showers and sinks. Usually said equipment maintains residual moisture that provides excellent conditions for bacterial growth and can therefore also cause bacterial based malodor.

WO 2011/098579 concerns bacterial deoxyribonuclease formulations and methods for biofilm disruption and prevention. Thus, said formulations may control bacterially caused malodors. However, the synthesis and purification of said nuclease polypeptides is time consuming and expensive.

Therefore, there is a long-standing need for the provision of a formulation that is able to control bacterial growth and malodor in long term and can be produced with low costs and minimal efforts.

In the present invention, the inventors surprisingly found that Bacillus strains (i) have extracellular enzyme activity, (ii) provide odor control, in particular they reduce or avoid malodors emerging in milk samples, (iii) demonstrate long term cleaning effects on hard surfaces and (iv) are able to survive in a wide pH range. Thus, the Bacillus strains of the present invention can be used without further ingredients or in combination with additional cleaning components to control malodors and/or to provide long term cleaning effects caused by microbial growth without the prior need of laborious and expensive protein purification. For the application in cleaning and malodor preventing formulations the Bacillus strains may be used in their sporulated forms.

### BRIEF SUMMARY OF THE INVENTION

In a first aspect, the problem underlying the present invention is solved by a Bacillus strain having extracellular hydrolase activity and demonstrating reduced malodor after incubation for at least 5 days for at least 25 °C in a lactose-free whole milk sample compared to a non-inoculated lactose-free whole milk sample.

In a preferred embodiment, the hydrolase activity is selected from protease activity, amylase activity, lipase activity, mannanase activity, cellulase activity or combinations thereof.

In a further preferred embodiment, the incubation is for at least 7 days for at least 30 °C. In preferred embodiments, said strain has the ability to form a spore and preferably is in the form of a spore.

The invention is also directed to preferred embodiments in which said strain is selected from the group of Bacillus species consisting of Bacillus methylotrophicus, Bacillus subtilis and Bacillus amyloliquefaciens.

In preferred embodiments the invention is directed to a Bacillus strain of the invention
i) as deposited under accession number DSM 34306 or a mutant thereof having all of the identifying characteristics as described above;
ii) as deposited under accession number DSM 34307 or a mutant thereof having all of the identifying characteristics as described above;
iii) as deposited under accession number DSM 34304 or a mutant thereof having all of the identifying characteristics as described above; or
iv) as deposited under accession number DSM 34305 or a mutant thereof having all of the identifying characteristics as described above.

In preferred embodiments the invention is directed to a Bacillus strain of the invention, wherein
i) the strain as deposited under accession number DSM 34306 comprises 16S rDNA having the sequence of SEQ ID NO:1;
ii) the strain as deposited under accession number DSM 34307 comprises 16S rDNA having the sequence of SEQ ID NO:2;
iii) the strain as deposited under accession number DSM 34304 comprises 16S rDNA having the sequence of SEQ ID NO:3; or
iv) the strain as deposited under accession number DSM 34305 comprises 16S rDNA having the sequence of SEQ ID NO:4.

In preferred embodiments the invention is directed to a Bacillus strain of the invention, wherein
i) the mutant of the strain as deposited under accession number DSM 34306 comprises 16S rDNA having at least 98% sequence identity to SEQ ID NO:1;
ii) the mutant of the strain as deposited under accession number DSM 34307 comprises 16S rDNA having at least 98% sequence identity to SEQ ID NO:2;
iii) the mutant of the strain as deposited under accession number DSM 34304 comprises 16S rDNA having at least 98% sequence identity to SEQ ID NO:3; or
iv) the mutant of the strain as deposited under accession number DSM 34305 comprises 16S rDNA having at least 98% sequence identity to SEQ ID NO:4.

In a second aspect, the present invention is directed to a composition comprising at least one Bacillus strain of the invention and at least one additional component.

In a preferred embodiment, the composition is a detergent composition, more preferably a laundry detergent composition, a hard surface cleaning detergent composition, a sanitary or kitchen cleaner or an anti-malodor product.

In a further preferred embodiment, the composition comprises one or more surfactants and/or one or more builders and/or one or more enzymes and/or one or more preservative agents, preferably one or more surfactants and/or one or more solvents.

In a third aspect, the present invention is directed to a method to improve detergency of a detergent formulation by adding at least one Bacillus strain of the invention to a detergent formulation.

In a fourth aspect, the present invention is directed to a cleaning method comprising contacting at least one Bacillus strain of the invention or a composition of the invention with an object that requires cleaning, preferably a laundry or a hard surface household item.

In a fifth aspect, the present invention is directed to a method to reduce malodor comprising contacting at least one Bacillus strain of the invention or a composition of the invention with an object that requires cleaning, preferably a laundry or a hard surface household item.

In a sixth aspect, the present invention is directed to a method to control or reduce malodor-causing microorganisms comprising contacting at least one Bacillus strain of the invention or a composition of the invention with an object or with a medium on/in which said microorganisms are present or grow.

In a seventh aspect, the present invention is directed to the use of a Bacillus strain as deposited under accession number DSM 34306, DSM 34307, DSM 34304 or DSM 34305 for malodor inhibition.

### BRIEF DESCRIPTION OF THE FIGURES

The present invention is further illustrated by the following figures and non-limiting sequences and examples.

FIG. 1 shows growth and spore formation of eight test strains, namely Bacillus methylotrophicus (comprising SEQ ID NO:1), Bacillus subtilis (comprising SEQ ID NO:2), Bacillus amyloliquefaciens (comprising SEQ ID NO:3), Bacillus amyloliquefaciens (comprising SEQ ID NO:4), Paenibacillus sp., Paenibacillus glycanilyticus, Paenibacillus amylolyticus and Gulosibacter faecalis after 72 h cultivation in BA055 medium.

FIG. 2 shows evaluation of four different enzyme activities on agar plates for the above-mentioned bacterial strains.

FIG. 3 shows the score values for the categories spore count, enzyme score and odor control as well as the total score for eight bacterial strains tested.

FIG. 4 shows DM-03 (shepherd's pie) CFT-tiles that were coated with spore formulations of the indicated strains for 7 days and 30 °C without mechanical cleaning and rinsed with dH₂O to remove soil. CTRL: surfactant without spores or water without spores.

FIG. 5 A shows spore viability of selected strains of the invention (Bacillus methylotrophicus (comprising SEQ ID NO:1), Bacillus subtilis (comprising SEQ ID NO:2), Bacillus amyloliquefaciens (comprising SEQ ID NO:3), Bacillus amyloliquefaciens (comprising SEQ ID NO:4)) in 60% glycerol solutions adjusted to pH 5 (grey+clear), 7 (white+dots) and 9 (black+stripes) stored at room temperature. No significant decrease in viability was observed for any strain.

FIG. 5 B shows a bright-field microscopy of Bacillus amyloliquefaciens (comprising SEQ ID NO:3) stored at pH 9 after 14 days (100× magnification) showing solely spores without any vegetative cells.

FIG. 6 shows the comparison of inventive strains with reference strains via sensory malodor determination and via HPLC measurement.

### DETAILED DESCRIPTION OF THE INVENTION

In the present invention, the inventors surprisingly found that certain Bacillus strains offer the combination of good sporulation and spore viability, sufficient extracellular enzyme activity, for example protease, lipase, amylase and mannanase activity and having the ability to control malodors caused by microorganisms, in particular they reduce or avoid malodors emerging in milk samples. These new Bacillus strains can be used in (hard surface) cleaning, ADW, malodor inhibiting and detergent formulations to provide cleaning effects and to control malodors. In specific embodiments, these Bacillus strains are selected from the group consisting of Bacillus methylotrophicus, Bacillus subtilis and Bacillus amyloliquefaciens.

In a first aspect, the problem underlying the present invention is solved by a Bacillus strain having extracellular hydrolase activity and demonstrating reduced malodor after incubation for at least 5 days for at least 25 °C in a lactose-free whole milk sample compared to a non-inoculated lactose-free whole milk sample.

The terms “Bacillus” and “Bacillus strain”, as interchangeably used herein, refer to a genus of Gram-positive, rod-shaped bacteria which are members of the division Firmicutes. Under stressful environmental conditions, the Bacillus bacteria produce oval endospores that can stay dormant for extended periods. Bacillus bacteria may be characterized and identified based on the nucleotide sequence of their 16S rRNA or a fragment thereof (e.g. approximately a 1000 nt, 1100 nt, 1200 nt, 1300 nt, 1400 nt, or 1500 nt fragment of 16S rRNA or rDNA nucleotide sequence). At the date of filing the genus Bacillus comprises 266 species and may include, but is not limited to B. acicliceler, B. acidicola, B. cidiproducens, B. aeoliiis, B. aerius, B. aerophilus, B. agaradaerens, B. aidingensis, B. akibai, B. alcalophilus, B. algicola, B. alkalinilriticilis, B. alkalisediminis, B. alkalitelhtris, B. allitudinis, B. alveayuensis, B. amyloliquefaciens, B. anthracis, B. aquimaris, B. arsenicus, B. aryabhatiai, B. asahii, B. atrophaeus, B. aurantiacus, B. azotoformans, B. badius, B. barharicus, B. baiaviensi, B. beijingensis, B. beiizoevorans, B. beveridgei, B. bogoriensis, B. boroiiiphilus, B. bilianolivoralis, B. canaveralius, B. carboniphilus, B. cecembensis, B. cellulosilyiiciis, B. cereiis, B. chagarmorensis, B. chwigangensis, B. cibi, B. circillans, B. clarkii, B. clausii, B. coagiilaiis, B. coahtiilensis, B. cohiii, B. decisifroiidis. B. decolor ationis, B. drenlensis, B. farraginis, B. faslidiosus, B. jirmus, B. flexus, B. foramrms, B. fordii, B. fords, B. fu arioli, B. funiculus, B. galactosidilylicus, B. galliciensis, B. geladni, B. gibsonii, B. giiisengi, B. giiisengihitini, B. graminis, B. halmapalus, B. halochares, B. halodurans, B. hemicellulosilyticus, B. herberlslcinensis, B. Iwrikoshi. B. lwrneckiae, B. horti. B. hominis, B. hwajinpoensis, B. idriensis, B. indicus, B. infantis, B. infermis, B. isabeliae, B. isronensis, B. jeolgali, B. koreensis, B. korlensis, B. kribbensis, B. krulwichiae, B. lehensis, B. lenlus, B. licheniformis, B. liloralis, B. locisalis, B. lucifereiisis, B. luleolus, B. macauensis, B. inacyae, B. mannaiiilylicus, B. marisflavi, B. marmarcnsis, B. inassilieiisis, B. inegaleiiu, B. methanolicits, B. methylotrophicus, B. mojavensis, B. muralis, B. Imirimardni, B. mycoidcs, B. iaielisis, B. iicmhaiisedimrms, B. nealsoiiii, B. neizhouensis, B. niabensis, B. iiiacini, B. novalis, B. oceaiiisediminis, B. odysseyi, B. okhensis, B. okiihideiisis, B. oleronius, B. oshimensis, B. panaciterrae, B. paiagoiensis, B. persepolensis, B. plakorlidis, B. pocheonensis, B. polygoni, B. pseudoalcaliphilus, B. pseiidofirnnis, B. pseudomycoides, B. psychrosaccharolyticus, B. pumilus, B. qingdaonensis, B. rigid, B. niris, B. safensis, B. salarhts, B. saliphilus, B. schlegelii, B. selenatarsenatis, B. selentireducens, B. seohaeanensis, B. shacklelonii, B. siamensis, B. simplex, B. siralis, B. smithii, B. soli, B. solisalsi, B. sonoreiisis, B. sporolhermodiirons, B. stralosphericits, B. subteiraneus, B. subtilis, B. taeansis, B. tequilensis, B. thermanlaicticus, B. thermoamylovorans, B. thermocloacae, B. thermolactis, B. thioparans, B. thuringiensis, B. tripoxylicola, B. tusciae, B. vallismortis, B. vedderi, B. vietnamensis, B. vireti, B. wakoensis, B. weienstephanensis, B. xiaoxieiisis, and mixtures or blends thereof. A more complete list of Bacillus species can be found under the following internet address and is part of this description by reference: https://lpsn.dsmz.de/genus/bacillus. The invention is also directed to preferred embodiments in which said strain is selected from the group of Bacillus species consisting of Bacillus methylotrophicus, Bacillus subtilis and Bacillus amyloliquefaciens.

The term “strain”, as used herein, refers to a genetic variant, a subtype or a culture within a biological species. In more preferred embodiments, the strain is naturally occurring or isolated from a natural sample, such as a soil, water, air or biological sample.

“Malodor”, as used herein, refers to unpleasant odor directly or indirectly caused by naturally occurring bacteria and other microorganisms, such as fungi and archaea. A malodor is considered to be directly caused by a microorganism when the malodor is caused by one or more metabolic intermediate and/or end products of the microorganism. Further, the microorganisms, specifically bacteria, can form biofilms. Molecules surrounding the biofilms and having an unpleasant odor can stick to the biofilm and accumulate. Thus, (bacterial) biofilms can be the origin of unpleasant smell of molecules that were not synthesized by the bacteria (indirect cause of malodor). The term “reduced malodor”, as used herein, refers to a decreased amount of the unpleasant odor or its total absence. In preferred embodiments, the malodor is reduced to at least 10%, at least 20%, at least 30%, at least 40%, at least 50%, at least 60%, at least 70%, at least 80%, at least 90%, at least 95% or at least 99% of the original malodor. The appearance of malodor may be measured by a smelling test or by using analytical instruments. The use of analytical instruments is preferred in cases when the chemical molecule(s) responsible for the malodor is/are known. In further embodiments, malodor refers to no or an insignificant amount of butyric acid under the conditions described herein, namely incubation of the inventive strain for at least 5 days for at least 25 °C in a lactose-free whole milk sample. In this set-up the butyric acid concentration may be below 0.2 g/L, preferably below 0.1 g/L, more preferably below 0.05 g/L and most preferred no butyric acid will be detectable using high-performance liquid chromatography (HPLC) measurement. Other methods to investigate the milk sample and detect metabolites of the microorganisms, such as butyric acid, are well-known to the skilled person and include, but are not limited to mass spectrometry-based methods, surface-enhanced Raman spectroscopy (SERS), nuclear magnetic resonance spectroscopy (NMR), surface plasmon resonance spectroscopy (SPRS) etc.

The terms “incubating” and “incubation”, as used herein, refer to a process of mixing a Bacillus strain of the invention with milk and allowing them to interact under conditions defined below. The Bacillus strain may be in fluid form, namely a bacteria dissolved in culture medium, or in solid form as a spore or vegetive Bacillus taken from a solid culture plate. The incubation of the bacterial strain and the milk may occur for at least 5, at least 6 or at least 7 days. The incubation temperature is at least 18 °C, at least 20 °C, at least 23 °C, at least 25 °C, at least 26 °C, at least 27 °C, at least 28 °C, at least 29 °C or at least 30 °C. The incubation temperature may be not more than 50 °C, not more than 45 °C, not more than 40 °C, not more than 38 °C, not more than 35 °C or not more than 33 °C. Preferably, the incubation temperature is constant. Preferred embodiments are directed to the incubation of the Bacillus strain of the invention and the milk for 5 days at 25-40 °C or 6 days at 25-40 °C or 7 days at 25-40 °C. In preferred embodiments, the Bacillus strain of the invention and the milk are incubated for at least 5 days for at least 25 °C, more preferably for at least 7 days for at least 30 °C. In even more preferred embodiments, the Bacillus strain of the invention and the milk are incubated for 5 days for 25 °C, more preferably for 7 days for 30 °C. “Non-inoculated”, as used herein, means that the milk sample is not consciously brought into contact with a bacterial strain of interest those ability to reduce malodor should be tested.

However, the bacteria and other microorganisms that cause milk spoilage will be found in this milk sample. Moreover, “lactose-free whole milk sample”, as used herein, refers to milk that contains all is natural fat content. This raw milk may have a fat content of at least 3%, at least 3.5%, at least 4%, at least 4.5%, at least 5% or at least 5.5%. The milk may originate from a mammal, preferably a cow, goat, sheep, or buffalo, more preferably cow. In addition, the milk is substantially free of lactose. In more detail, the term “lactose-free”, as used herein, is to be understood to include milk that comprises less than 0.20% w/w of lactose on a liquid milk basis, more specifically, less than 0.15% w/w of lactose on a liquid milk basis, even more specifically less than 0.10% w/w of lactose on a liquid milk basis, and even more specifically between 0.01%-0.05% w/w of lactose on a liquid milk basis and even more specifically not more than 0.01% of lactose on liquid milk basis. It noted that measurements to obtain the lactose content of a milk sample may be prepared on a dry solid milk basis and subsequently are converted to a liquid milk basis. In even more preferred embodiments, the milk is not sterilized.

The term “(extracellular) hydrolase activity”, as used herein, refers to the catalytic activity of a group of enzymes that use water to break a chemical bond, which typically results in dividing a larger molecule into smaller molecules. The term “extracellular” refers in this context to enzymatic activity that can be measured outside in the surroundings of the bacterium. This extracellular activity is caused by enzymes that the produced in the bacterium and then transported outside the bacterial cell. Some common examples of hydrolase enzymes are esterases including lipases, phosphatases, glycosidases, peptidases, and nucleosidases.

Hydrolases are classified as EC 3 in the EC number classification of enzymes. Hydrolases can be further classified into several subclasses, based upon the bonds they act upon: EC 3.1: ester bonds (esterases: nucleases, phosphodiesterases, lipase, phosphatase), EC 3.2: sugars (DNA glycosylases, glycoside hydrolase), EC 3.3: ether bonds, EC 3.4: peptide bonds (Proteases/peptidases), EC 3.5: carbon-nitrogen bonds, other than peptide bonds, EC 3.6 acid anhydrides (acid anhydride hydrolases, including helicases and GTPase), EC 3.7 carbon-carbon bonds, EC 3.8 halide bonds, EC 3.9: phosphorus-nitrogen bonds, EC 3.10: sulphur-nitrogen bonds, EC 3.11: carbon-phosphorus bonds, EC 3.12: sulfur-sulfur bonds and EC 3.13: carbon-sulfur bonds. In a preferred embodiment, the hydrolase activity is selected from protease activity, amylase activity, lipase activity, mannanase activity, cellulase activity or combinations thereof.

The term “protease”, as used herein, refers to at least one protease that may be selected from serine proteases (EC 3.4.21). Serine proteases or serine peptidases are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5), and subtilisin. Subtilisin is also known as subtilopeptidase, e.g., EC 3.4.21.62, the latter hereinafter also being referred to as “subtilisin”. In preferred embodiments, the protease exhibiting extracellular activity is a subtilisin according to EC 3.4.21.62. The activity of the protease may be determined on agar plates or in liquid enzyme assays. Assays to measure protease activity are well known in the art and can include, without being limited to colorimetric assays, mass spectrometry-based assays and fluorescence resonance energy transfer (FRET) assays. These assay types may also be used to determine the activity of the lipase, amylase, mannanase as well as the cellulase activity. In preferred embodiments, the protease assay uses milk powder as a substrate and the protease activity is at least 0.002, at least 0.005, at least 0.008, at least 0.01, at least 0.012 or at least 0.015 AU/min*mL.

“Amylases” according to the invention (alpha and/or beta) include those of bacterial origin (EC 3.2.1.1 and 3.2.1.2, respectively). Preferably, amylases are selected from the group of alpha-amylases (EC 3.2.1.1). Chemically modified or protein engineered mutants are included. Amylases according to the invention have “amylolytic activity” or “amylase activity” involving (endo)hydrolysis of glucosidic linkages in polysaccharides. alpha-amylase activity may be determined by assays for measurement of alpha-amylase activity which are known to those skilled in the art. Examples for assays measuring alpha-amylase activity are: alpha-amylase activity can be determined by a method employing Phadebas tablets as substrate (Phadebas Amylase Test, supplied by Magle Life Science). Starch is hydrolyzed by the alpha-amylase giving soluble blue fragments. The absorbance of the resulting blue solution, measured spectrophotometrically at 620 nm, is a function of the alpha-amylase activity. The measured absorbance is directly proportional to the specific activity (activity/mg of pure alpha-amylase protein) of the alpha-amylase in question under the given set of conditions. Alpha-amylase activity can also be determined by a method employing the Ethyliden-4-nitrophenyl-alpha-D-maltoheptaosid (EPS). D-maltoheptaoside is a blocked oligosaccharide which can be cleaved by an endo-amylase. Following the cleavage, the alpha-glucosidase included in the kit to digest the substrate to liberate a free PNP molecule which has a yellow color and thus can be measured by visible spectophotometry at 405 nm. Kits containing EPS substrate and alpha-glucosidase is manufactured by Roche Costum Biotech (cat. No. 10880078103). The slope of the time dependent absorption-curve is directly proportional to the specific activity (activity per mg enzyme) of the alpha-amylase in question under the given set of conditions. In preferred embodiments, the amylase assay uses corn starch stained with Lugolsch solution or red starch as a substrate and the amylase activity is at least 1.2, at least 1.5, at least 2.0, at least 2.5, at least 3.0, at least 3.5, at least 4.0, at least 4.5 or at least 4.7 U/mL.

“Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases include those of bacterial or fungal origin. The methods for determining lipolytic activity are well-known in the literature (see e.g. Gupta et al. (2003), Biotechnol. Appl. Biochem. 37, p. 63-71). E.g. the lipase activity may be measured by ester bond hydrolysis in the substrate para-nitrophenyl palmitate (pNP-Palmitate, C:16) and releases pNP which is yellow and can be detected at 405 nm. “Lipolytic activity” means the catalytic effect exerted by a lipase, which may be provided in lipolytic units (LU). For example, 1 LU may correspond to the amount of lipase which produces 1 pmol of titratable fatty acid per minute in a pH stat. under the following conditions: temperature 30 °C; pH=9.0; substrate may be an emulsion of 3.3 wt. % of olive oil and 3.3% gum arabic, in the presence of 13 mmol/I Ca2+ and 20 mmol/I NaCl in 5 mmol/I Tris-buffer. In preferred embodiments, the lipase assay uses tributyrin or methylumbelliferyl heptanoate as a substrate and the amylase activity is at least 48, at least 100, at least 200, at least 300, at least 400, at least 500, at least 600, at least 700 or at least 790 U/mL.

The term “mannanase”, as used herein, refers to a polypeptide selected from the group of mannan degrading enzyme. At least one mannan degrading enzyme may be selected from β-mannosidase (EC 3.2.1.25), endo-1,4-β-mannosidase (EC 3.2.1.78), and 1,4-β-mannobiosidase (EC 3.2.1.100). Preferably, at least one mannan degrading enzyme is selected from the group of endo-1,4-β-mannosidase (EC 3.2.1.78), a group of enzymes which may be called endo-β-1,4-D-mannanase, β-mannanase, or mannanase herein. A polypeptide having mannan degrading activity or mannanase activity may be tested for according to standard test procedures known in the art, such as by applying a solution to be tested to 4 mm diameter holes punched out in agar plates containing 0.2% AZCL galactomannan (carob), i.e. substrate for the assay of endo-1,4-beta-D-mannanase available as CatNo. I-AZGMA from the company Megazyme (Megazyme's Internet address: http://www.megazyme. com/Purchase/index. html). Mannan degrading activity may alternatively be tested in a liquid assay using carob galactomannan dyed with Remazol Brilliant Bue as described in McCleary, B. V. (1978). Carbohydrate Research, 67(1), 213-221. Another method for testing mannan degrading activity uses detection of reducing sugars when incubated with substrate such as guar gum or locust bean gut—for reference see Miller, G. L.Use of Dinitrosalicylic Acid Reagent for Determination of Reducing Sugars. Analytical Chemistry 1959; 31: 426-428. In preferred embodiments, the mannanase assay uses Locust Bean Gum as a substrate and the amylase activity is at least 1.2, at least 1.5, at least 2.0, at least 2.5, at least 3.0, at least 3.5, at least 4.0, at least 4.5 or at least 4.7 U/mL.

“Cellulases”, “cellulase enzymes” or “cellulolytic enzymes” according to the invention are enzymes involved in hydrolysis of cellulose, i.e having “cellulolytic activity” or “cellulase activity”. At least one cellulase of the invention may be selected from cellobiohydrolase (1,4-P-D-glucan cellobiohydrolase, EC 3.2.1.91), endo-ss-1,4-glucanase (EC 3.2.1.4) and ss-glucosidase (EC 3.2.1.21). Endoglucanases of EC class 3.2.1.4 may be named endoglucanase, endo-1,4-ss-D-glucan 4-glucano hydrolase, endo-1,4-beta-glucanase, carboxymethyl cellulase, and beta-1,4-glucanase. Assays for measurement of “cellulase activity” or “cellulolytic activity” are known to those skilled in the art. For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937). Cellulolytic activity may be provided in units per gram enzyme. For example, 1 unit may liberate 1.0 μmole of glucose from cellulose in one hour at pH 5.0 at 37 °C (2 hour incubation time). In preferred embodiments, the cellulase activity is at least 1.7, at least 3.0, at least 4.0, at least 10.0, at least 20.0, at least 25.0, at least 30.0, at least 50.0 or at least 58.0 U/mL.

In a further preferred embodiment, the incubation is for at least 7 days for at least 30 °C.

In preferred embodiments, said strain has the ability form a spore and preferably is in the form of a spore. The term “bacillus cell” refers to both the spore and the vegetative bacillus cell. The term “spore” or “bacillus spore”, as interchangeably used herein, refers to a spore which can be characterized as a resting, solid, non-reproductive structure produced by bacilli. The primary function of spores is, in general, to ensure the survival of bacteria during periods when external conditions become unfavorable. They are also resistant to ultraviolet radiation and gamma radiation, desiccation, lysozyme, temperature, starvation and chemical methods of exposure. Spores are usually found in soil and water, where they can persist for a long time. The spore membrane is impervious to many toxic molecules and may also contain enzymes involved in spore growth. The spore nucleus contains the usual cellular structures, such as DNA and ribosomes, but it is metabolically inactive. When the environment becomes unfavorable for the bacterium, it can begin the sporulation process, which takes about 8 hours. In preferred embodiments, the spore is an oval spore. The term “vegetative bacillus cell” refers to functional vegetative bacillus cells that can divide and produce more vegetative cells. The vegetative cells also have an active metabolism.

In preferred embodiments the invention is directed to a Bacillus strain of the invention
i) as deposited under accession number DSM 34306 or a mutant thereof having all of the identifying characteristics as described above;
ii) as deposited under accession number DSM 34307 or a mutant thereof having all of the identifying characteristics as described above;
iii) as deposited under accession number DSM 34304 or a mutant thereof having all of the identifying characteristics as described above; or
iv) as deposited under accession number DSM 34305 or a mutant thereof having all of the identifying characteristics as described above.

The term “mutant”, as used herein, refers to a mutated or deleted form of a gene or another section of the genomic sequence. Preferably, the mutation occurs in the sequence of the 16S rDNA. The mutation can be associated with a functional amendment of the nucleic acid or the corresponding protein. The functional amendment may be a loss of function or may increase the function, e.g. the binding activity of a nucleic acid and/or protein towards another nucleic acid and/or protein. Thus, a mutation in the 16S rDNA may cause reduced binding of the encoded 16S rRNA molecule towards the Shine-Dalgarno sequence or towards proteins of the 30S subunit of the ribosome, such as S2-S20.

The term “identifying characteristics as described above” refers to the characteristics of the Bacillus strain of the invention (i) to provide extracellular enzyme activity; (ii) to reduce malodor and (iii) to form a spore, all as described in more detail above.

In preferred embodiments the invention is directed to a Bacillus strain of the invention, wherein
i) the strain as deposited under accession number DSM 34306 comprises 16S rDNA having the sequence of SEQ ID NO:1;
ii) the strain as deposited under accession number DSM 34307 comprises 16S rDNA having the sequence of SEQ ID NO:2;
iii) the strain as deposited under accession number DSM 34304 comprises 16S rDNA having the sequence of SEQ ID NO:3; or
iv) the strain as deposited under accession number DSM 34305 comprises 16S rDNA having the sequence of SEQ ID NO:4.

As used herein, “16S rDNA” refers to the gene encoding the 16S ribosomal RNA (16S rRNA DNA) constituted of about 1500 nucleotides, which is the main component of the small prokaryotic ribosomal subunit (30S). Also, as explained further below, the bacteria of the invention described herein may have a 16S rDNA sequence with a certain sequence identity to the SEQ ID Nos. as listed below.

In preferred embodiments the invention is directed to a Bacillus strain of the invention, wherein
i) the mutant of the strain as deposited under accession number DSM 34306 comprises 16S rDNA having at least 98% sequence identity to SEQ ID NO:1;
ii) the mutant of the strain as deposited under accession number DSM 34307 comprises 16S rDNA having at least 98% sequence identity to SEQ ID NO:2;
iii) the mutant of the strain as deposited under accession number DSM 34304 comprises 16S rDNA having at least 98% sequence identity to SEQ ID NO:3; or
iv) the mutant of the strain as deposited under accession number DSM 34305 comprises 16S rDNA having at least 98% sequence identity to SEQ ID NO:4.

In a preferred embodiment, the term “mutant”, as used herein, may refer to at least one fragment or the full length sequence of SEQ ID Nos. 1, 2, 3 or 4. Such a fragment or full length sequence comprises or encodes for a 16S rRNA having at least 100, at least 200, at least 300, at least 500, at least 700, at least 900, at least 1100, at least 1300 or at least 1500 successive nucleotides of the original sequence. In another preferred embodiment, the term “mutant” relates not only to at least one fragment, but also a nucleotide sequence or a fragment thereof that are at least 98, 98.2, 98.4, 98.6, 98.8, 99.0, 99.2, 99.4, 99.5, 99.6, 99.7, 99.8 or 99.9% identical to the reference nucleotide sequence referred to. Known methods comprise various methods that may be used to align two given nucleic acid or amino acid sequences and to calculate the degree of identity, see for example Arthur Lesk (2008), Introduction to bioinformatics, Oxford University Press, 2008, 3rd edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007): Clustal Wand Clustal X version 2.0. Bioinformatics, 23, 2947-2948) is used applying default settings. The mutant has at least 98% sequence identity to the sequence according to SEQ ID Nos. 1, 2, 3 or 4. In preferred embodiments, the mutant has at least 99% sequence identity to the sequence according to SEQ ID Nos. 1, 2, 3 or 4. In even more preferred embodiments, the mutant has at least 99.5% sequence identity to the sequence according to SEQ ID Nos. 1, 2, 3 or 4.

In addition to the feature that the mutants of the strains as deposited under accession numbers DSM 34306, DSM 34307, DSM 34304 and DSM 34305 comprise 16S rDNA having at least 98% sequence identity to SEQ ID Nos. 1, 2, 3 and 4, respectively, these mutants have a genome that contains at least one (circulated) nucleotide strand that hybridizes to the complementary nucleotide strand of the corresponding wildtype genome under high stringency conditions. Preferably, both strands of the mutant hybridize to their corresponding wildtype strands, respectively, under stringent conditions. It is noted that these mutants still possess the “identifying characteristics as described above”, namely (i) to provide extracellular enzyme activity; (ii) to reduce malodor and (iii) to form a spore, all as described in more detail above. In more preferred embodiments, strain as deposited under accession number DSM 34306 comprises 16S rDNA having at least 98% sequence identity to SEQ ID Nos. 1 and has a genome that contains at least one (circulated) nucleotide strand that hybridizes to the complementary nucleotide strand of the genome of the strain as deposited under accession number DSM 34306 under high stringency conditions. Alternatively, in more preferred embodiments, strain as deposited under accession number DSM 34307 comprises 16S rDNA having at least 98% sequence identity to SEQ ID Nos. 2 and has a genome that contains at least one (circulated) nucleotide strand that hybridizes to the complementary nucleotide strand of the genome of the strain as deposited under accession number DSM 34307 under high stringency conditions. Alternatively, in more preferred embodiments, strain as deposited under accession number DSM 34304 comprises 16S rDNA having at least 98% sequence identity to SEQ ID Nos. 3 and has a genome that contains at least one (circulated) nucleotide strand that hybridizes to the complementary nucleotide strand of the genome of the strain as deposited under accession number DSM 34304 under high stringency conditions. Alternatively, in more preferred embodiments, strain as deposited under accession number DSM 34305 comprises 16S rDNA having at least 98% sequence identity to SEQ ID Nos. 4 and has a genome that contains at least one (circulated) nucleotide strand that hybridizes to the complementary nucleotide strand of the genome of the strain as deposited under accession number DSM 34305 under high stringency conditions. More preferably, the hybridization of the mutants is tested under very high stringency conditions. For nucleic acids, similar sequences can also be determined by hybridization using respective stringency conditions. The term “high stringency conditions” means for probes of at least 100 nucleotides in length, prehybridization and hybridization at 42 °C in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65 °C. The term “very high stringency conditions” means for probes of at least 100 nucleotides in length, prehybridization and hybridization at 42 °C in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70 °C.

In a second aspect, the present invention is directed to a composition comprising at least one Bacillus strain of the invention and at least one additional component. Preferably, the additional component is selected from group consisting of one or more surfactants and/or one or more builders and/or one or more preservative agents and/or one or more enzymes and/or one or more solvents, preferably one or more surfactants and/or one or more solvents. “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric.

The term “builder,” as used herein, may refer to a compound or substance that may stabilize another compound or composition by neutralizing the compound or composition. Further, builders may sequester calcium and magnesium hardness ions that might otherwise bind with and render the auxiliary surfactants or co-surfactants less effective. A “sequestering builder”, as used herein, is different from a precipitating builder in that no significant amount of precipitate is formed when the builder is used in an amount sufficient to combine with all of the calcium ions in an aqueous solution with 7° dH hardness (German hardness) initially at neutral pH. A “strong builder” is classified as high efficiency chelators that can bind the divalent cations such as Ca²⁺ strongly with a logarithmic stability constant (Log KCa) of the cation/chelator complex of above 4, particular above 5, above 6 or above 7. The stability constants are determined at an ionic strength of 0.1 M and at a temperature of 25 °C. A “strong sequestering builder” combines both of the above-mentioned properties. The solvent may be water and/or an organic solvent. The organic solvent may be a water-miscible solvent. The organic solvent may be one or more selected from the group consisting of glycerol, propanediol, polypropylene glycol, and polyethylene glycol.

The term “preservative agent”, as used herein, refers to an agent that is a chemical compound that controls microorganisms or inhibits their growth or reproduction or, dependent on their concentration, even kills microorganisms. A preservative may be an antimicrobial agent which may be added to aqueous products and compositions to maintain the original performance, characteristics and integrity of the products and compositions by inhibiting the growth of microorganisms or even killing them dependent on the concentration of the preservative agent.

The composition/formulation may contain one or more preservatives as listed in patent WO2021/115912 A1 (“Formulations comprising a hydrophobically modified polyethyleneimine and one or more enzymes”) on pages 35 to 39. Especially of interest for the cleaning compositions and fabric and home care products and specifically in the laundry formulations are any of the following preservatives: 4,4′-dichloro 2-hydroxydiphenyl ether (further names: 5-chloro-2-(4-chlorophenoxy) phenol, Diclosan, DCPP), Tinosan® HP 100 (commercial product of BASF SE containing 30% of active 4,4′-dichoro 2-hydroxydiphenylether); 2-Phenoxyethanol (further names: Phenoxyethanol, Methylphenylglycol, Phenoxetol, ethylene glycol phenyl ether, Ethylene glycol monophenyl ether, 2-(phenoxy) ethanol, 2-phenoxy-1-ethanol); 2-bromo-2-nitropropane-1,3-diol (further names: 2-bromo-2-nitro-1,3-propanediol, Bronopol); Glutaraldehyde (further names: 1-5-pentandial, pentane-1,5-dial, glutaral, glutar-dialdehyde); Glyoxal (further names: ethandial, oxylaldehyde, 1,2-ethandial); 2-butyl-benzo[d]isothiazol-3-one (“BBIT”); 2-methyl-2H-isothiazol-3-one (“MIT” ”); 2-octyl-2H-isothiazol-3-one (“OIT”); 5-Chloro-2-methyl-2H-isothiazol-3-one (“CIT” or “CMIT”); Mixture of 5-chloro-2-methyl-2H-isothiazol-3-one (“CMIT”) and 2-methyl-2H-isothiazol-3-one (“MIT”) (Mixture of CMIT/MIT); 1,2-benzisothiazol-3(2H)-one (“BIT”); Hexa-2,4-dienoic acid (trivial name “sorbic acid”) and its salts, e.g., calcium sorbate, sodium sorbate; potassium (E,E)-hexa-2,4-dienoate (Potassium Sorbate); Lactic acid and its salts; L-(+)-lactic acid; especially sodium lactate; Benzoic acid and salts of benzoic acid, e.g., sodium benzoate, ammonium benzoate, calcium benzoate, magnesium benzoate, MEA-benzoate, potassium benzoate; Salicylic acid and its salts, e.g., calcium salicylate, magnesium salicylate, MEA salicylate, sodium salicylate, potassium salicylate, TEA salicylate; Benzalkonium chloride, benzalkonium bromide, benzalkonium saccharinate; Didecyldimethylammonium chloride (“DDAC”); N-(3-aminopropyl)-N-dodecylpropane-1,3-diamine (“Diamine”); Peracetic acid; Hydrogen peroxide and formic acid. At least one preservative may be added to the inventive composition in a concentration of 0.001 to 10% relative to the total weight of the composition. Preferably, the composition contains 2-phenoxyethanol in a concentration of 0.1 to 2% or 4,4′-dichloro 2-hydroxydiphenyl ether (DCPP) in a concentration of 0.005 to 0.6%. Preferred preservative agents for hard surface cleaning composition are selected from 2-Phenoxyethanol, Bronopol and formic acid.

In addition, the at least one additional component may be an enzyme added in additional to the enzymes secreted by the Bacillus strain of the invention. In one embodiment, the enzyme, which is not derived from the strain of the invention, is classified as an oxidoreductase (EC 1), a transferase (EC 2), a hydrolase (EC 3), a lyase (EC 4), an isomerase (EC 5), or a ligase (EC 6). The EC-numbering is according to Enzyme Nomenclature, Recommendations (1992) of the Nomenclature Committee of the International Union of Biochemistry and Molecular Biology including its supplements published 1993-1999. Preferably, the enzyme is a hydrolase (EC 3). In a preferred embodiment, the enzyme, which is not derived from the strain of the invention, is selected from the group consisting of proteases, amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the enzyme is selected from the group consisting of proteases, amylases, lipases, cellulases, mannanases, xylanases, DNases, dispersins, pectinases, oxidoreductases, and cutinases, and combinations of at least two of the foregoing types. Most preferably, the enzyme, which is not derived from the strain of the invention, is a protease, preferably, a serine protease, more preferably, a subtilisin protease.

In a preferred embodiment, the composition is a detergent composition, more preferably a laundry detergent composition, a hard surface cleaning detergent composition, a sanitary or kitchen cleaner or an anti-malodor product. Preferably, it is a cleaning composition and/or fabric and home care product, comprising at least one Bacillus strain of the invention, as defined above, preferably for improved oily and fatty stain removal, preferably a laundry detergent formulation and/or a manual dish wash detergent formulation, more preferably a liquid laundry detergent formulation and/or a liquid manual dish wash detergent formulation and/or sanitary cleaner. In another preferred embodiment of the present invention, the cleaning composition may be used for soil removal of particulate stains and/or oily and fatty stains, and additionally for whiteness maintenance, preferably in laundry care. In another embodiment, the cleaning composition of the present invention is a hard surface cleaning composition that may be used for cleaning various surfaces such as hard wood, tile, ceramic, plastic, leather, metal, or glass.

In another embodiment, the cleaning composition of the present invention is a liquid or solid automatic dish wash detergent composition, preferably a solid automatic dish wash detergent composition, that may be used for cleaning dish ware, e.g., dish ware such as glasses.

In a third aspect, the present invention is directed to a method to improve detergency of a detergent formulation by adding at least one Bacillus strain of the invention to a detergent formulation.

In a fourth aspect, the present invention is directed to a cleaning method comprising contacting at least one Bacillus strain of the invention or a composition of the invention with an object that requires cleaning, preferably a laundry or a hard surface household item. The term “object (that requires cleaning)”, as used herein, refers to any object possessing a surface that attracts soil. In particular, the object can be one that has a flexible structure, such as laundry. However, in other preferred embodiments, the object is a household item having a solid and hard surface. Such objects include smaller movable objects, such as dishes, glasses, and tiles. Nonetheless, this group of objects also includes bigger, non-movable objects, specifically sanitary objects, such as toilets, showers and sinks or kitchen items, such as fridges, freezers, work benches or tops and storage areas. In preferred embodiments, the Bacillus strains of the invention are used in cleaning methods and formulations that are associated with the storage of food, in particular the storage of milk and milk or dairy products, such as, but not limited to, cream, butter, yogurt, cheese, condensed milk, kefir, milk powder, sour cream, creme fraiche, ymer, custard, chocolate, and ice cream.

In a fifth aspect, the present invention is directed to a method to reduce malodor comprising contacting at least one Bacillus strain of the invention or a composition of the invention with an object that requires cleaning, preferably a laundry or a hard surface household item.

In a sixth aspect, the present invention is directed to a method to control or reduce malodor-causing microorganisms comprising contacting at least one Bacillus strain of the invention or a composition of the invention with an object or with a medium on/in which said microorganisms is present or grows.

The term “malodor-causing microorganism”, as used herein, contains all forms of microorganisms, such as bacteria, archaea, fungi and protozoa that are directly or indirectly responsible for unpleasant odor. In preferred embodiments, these microorganisms are psychrotrophs. “Psychrophiles” or “cryophiles” are extremophilic organisms that are capable of growth and reproduction in low temperatures, ranging from −20 °C to 20 °C. They have an optimal growth temperature at 15 °C. Many such organisms are bacteria or archaea, but also some eukaryotes such as lichens, snow algae, phytoplankton, fungi, and wingless midges, are classified as psychrophiles.

The terms “medium”, as used herein, refer to a solution containing nutrients that nourish growing microorganisms, e.g., bacteria. Typically, these solutions provide essential and nonessential amino acids, vitamins, energy sources, lipids, and trace elements required by the cell for minimal growth and/or survival. The solution may also contain components that enhance growth and/or survival above the minimal rate. The solution has a pH and salt concentration allowing cell survival and proliferation.

In a seventh aspect, the present invention is directed to the use of a Bacillus strain as deposited under accession number DSM 34306, DSM 34307, DSM 34304 or DSM 34305 for malodor inhibition.

The following biological material has been deposited under the terms of the Budapest Treaty at Leibniz Institute, DSMZ-German Collection of Microorganisms and Cell Cultures GmbH, InhoffenstraBe 7B, 38124 Braunschweig, GERMANY and given the following accession number:

| Identification | Accession Number | Date of Deposit |
|---|---|---|
| Bacillus methylotrophicus | DSM 34306 | Sep. 2, 2022 |
| Bacillus subtilis | DSM 34307 | Sep. 2, 2022 |
| Bacillus amyloliquifaciens | DSM 34304 | Sep. 2, 2022 |
| Bacillus amyloliquifaciens | DSM 34305 | Sep. 2, 2022 |

Unless otherwise noted, the terms used herein are to be understood according to conventional usage by those of ordinary skill in the relevant art. Unless stated otherwise or apparent from the nature of the definition, the definitions apply to all compounds, methods and uses described herein.

Compositions of the invention comprise at least one microorganism as described herein. The microorganisms should be present in effective amounts. The terms “effective amount”, “effective concentration” or “effective dosage” are defined herein as the amount, concentration or dosage of one or more odor-controlling microbial strains that can inhibit the malodor caused by the odor causing organism or substances derived therefrom on articles, articles subjected to a cleaning machine or cleaning process, and/or cleaning machines. In preferred embodiments, the composition of the invention comprises 0.001-5% by weight Bacillus strain of the invention. The weight of the spores may be the weight of dry spores or wet biomass, preferably dry spores.

The compositions can be solid, semi-solid or gel-like, liquid (including spray) or an aerosol. They can be formulated in all types usual for the respective application, such as bars, powders, granulates, agglomerates, pastes, gels, solutions, emulsions, suspensions, etc. They can also be formulated as liquid composition imbibed in wipes or pads.

The compositions generally contain a carrier. In liquid, semi-solid or gel-like compositions, the carrier is or comprises a solvent, mostly water, (and/or) an alkanol (generally a C2-C3-alkanol, i.e. ethanol, n-propanol and/or isopropanol; these generally also act as wetting agents to allow a better wetting or penetration of the treated substrate by the composition; this latter effect is particularly useful if no surfactant is contained in the composition), an organic solvent different therefrom (details to such further solvents are given below in context with preferred embodiments of the composition) or a mixture thereof. In solid, semi-solid or gel-like compositions, the carrier is or comprises a solid carrier. In solid soaps, the soap component (e.g. the solid salt of long-chained fatty acids) is generally also the carrier.

Depending on the targeted use, the compositions generally comprise further components. Examples are pH adjusting agents, sequestrants, thickeners, antifreezing agents, antifoaming agents, colorants, or perfumes. Further details to such further components are given below in context with preferred embodiments of the composition.

In a preferred embodiment, the (liquid) composition comprises
(a) 0 to 2% by weight (=2 to 90<100 ppm), relative to the total weight of the composition, of the at least one preservative agent;
(b) 0.01-10% by weight, relative to the total weight of the composition of at least a nonionic or an anionic surfactant
(c) 0 to 10% by weight, relative to the total weight of the composition, of one or more surfactants;
(d) 0 to 90% by weight, relative to the total weight of the composition, of at least one C₂-C₃-alkanol;
(e) 0 to 10% by weight, relative to the total weight of the composition, of at least one organic solvent;
(f) 0 to 10% by weight, relative to the total weight of the composition, of at least one sequestrant;
(g) 0 to 10% by weight, relative to the total weight of the composition, of a further additive; and/or
(h) ad 100% by weight, but at least 30% by weight, relative to the total weight of the composition, of water.

Preferably, at least one of components (c) to (g) is present. More preferably, at least component (c) is present.

Surfactants (or surface-active compounds) (termed component (c) in the above and below embodiments) can be anionic, cationic, non-ionic or amphoteric (zwitterionic). Anionic, cationic, non-ionic and amphoteric surfactants are widely known in the art.

Anionic surfactants are, for example, of the sulfate, sulfonate or carboxylate type or mixed forms thereof. Examples are
alkyl sulfates (generally of the formula R—O—SO₃⁻M⁺, where R is a long-chained alkyl group, e.g. C₃-C₂₄-alkyl, and M⁺ is a cation equivalent, generally Na⁺, K⁺, NH4⁺, mono-, di- or triethanolammonium); e.g. sodium lauryl sulfate;
alkyl ether sulfates (generally of the formula R—(CH₂CH₂—_{O})ₓ—O—SO₃⁻M⁺, where R is a long-chained alkyl group, e.g. C₈-C₂₄-alkyl, x is 1-10 and M⁺ is a cation equivalent, generally Na⁺, K⁺, NH4⁺, mono-, di- or triethanolammonium); e.g. so-dium laureth sulfate (SLES);
alkylbenzenesulfonates (generally of the formula R—(C₆H₄)—SO₃⁻M⁺, where R is a long-chained alkyl group, e.g. C₃-C₂₄-alkyl, and M⁺ is a cation equivalent, generally Na⁺, K⁺, NH₄⁺, mono-, di- or triethanolammonium); e.g. sodium dodecylbenzene sulfonate;
olefin sulfonates (generally of the formula R—SO₃⁻M⁺, where R is a long-chained monoolefin group, e.g. C₁₂-C₂₄-alkenyl, and M⁺ is a cation equivalent, generally Na⁺, K⁺, NH₄⁺, mono-, di- or triethanolammonium); e.g. sodium C₁₄/C₁₆-α-olefin sulfonate;
alkane sulfonates (generally of the formula R—SO₃⁻M⁺, where R is a long-chained alkyl group, e.g. C₃-C₂₄-alkyl, and M⁺ is a cation equivalent, generally Na⁺, K⁺, NH₄⁺, mono-, di- or triethanolammonium); e.g. alkali metal or ammonium C₁₃-C₁₇ paraffin sulfonate;
sulfated monoglycerides (generally of the formula R—COO—CH₂—CH(OH)—CH₂—O—SO₃⁻M⁺, where R is a long-chained alkyl group, e.g. C₃-C₂₄-alkyl, and M⁺ is a cation equivalent, generally Na⁺, K⁺, NH₄⁺, mono-, di- or triethanolammonium); e.g. sodium cocomonoglyceride sulfate;
alkyl sulfosuccinates, e.g. disodium N-octadecylsulfosuccinamate, diammonium lauryl sulfosuccinate, tetrasodium N-(1,2-dicarboxyethyl)-N-octadecylsulfosuccinate; diamyl ester of sodium sulfosuccinic acid, dihexyl ester of sodium sulfosuccinic acid, or dioctyl ester of sodium sulfosuccinic acid;
acyl taurates, e.g. N-alkyltaurins, e.g. prepared by reacting dodecylamine with sodiumisethionate or N-acyltaurines obtained by the reaction of N-methyl taurine with fatty acids;
acyl isethionates (generally of the formula R—COO—CH₂CH₂—SO₃⁻M⁺, where R is a long-chained alkyl group, e.g. C₁₀-C₃₀-alkyl, and M⁺ is a cation equivalent, generally Na⁺, K⁺, NH₄⁺, mono-, di- or triethanolammonium); e.g. ammonium cocoyl isethionate, sodium cocoyl isethionate or sodium lauroyl isethionate;
alkyl glycerylether sulfonates (generally of the formula R—O—CH₂—CH(OH)—CH₂—SO₃⁻M⁺, where R is a long-chained alkyl group, e.g. C₃-C₂₄-alkyl, and M+ is a cation equivalent, generally Na⁺, K⁺, NH₄⁺, mono-, di- or triethanolammonium); e.g. cocoglyceryl ether sulfonate;
sulfonated fatty acids and sulfonate fatty acid methyl esters (generally of the formula R—CH(SO₃ M⁺)—COOH and R—CH(SO₃ M⁺)—COOCH₃, where R is a long-chained alkyl group, e.g. C₃-C₂₄-alkyl, and M+ is a cation equivalent, generally Na⁺, K⁺, NH₄⁺, mono-, di- or triethanolammonium); e.g. a-sulfonated coconut fatty acid or lauryl methyl ester;
acyl glutamates (generally of the formula R—CO—N(COOH)—CH₂CH₂—COO⁻M⁺, where R is a long-chained alkyl group, e.g. C₃-C₂₄-alkyl, and M+ is a cation equivalent, generally Na⁺, K⁺, NH₄⁺, mono-, di- or triethanolammonium); e.g. sodium lauroyl glutamate or sodium cocoyl glutamate;
acyl sarcosinates (generally of the formula R—CO—N(CH₃)—CH₂—COO⁻M⁺, where R is a long-chained alkyl group, e.g. C₃-C₂₄-alkyl, and M+ is a cation equivalent, generally Na⁺, K⁺, NH₄⁺, mono-, di- or triethanolammonium); e.g. sodium lauroyl sarcosinate, sodium cocoyl sarcosinate or ammonium lauroyl sarcosinate;
alkyl sulfoacetates,
fatty acid salts, generally derived from the saponification of oils or fats, e.g. from palm oil or tallow oil, and having from 8 to 24 carbon atoms in the alkyl/alkenyl moiety (thus containing, inter alia, oleate, linolate, palmitate, myristate, stearate etc.), where the counter cation is generally Na⁺, K⁺, NH₄⁺, mono-, di- or triethanolammonium;
alkyl and alkenyl ether carboxylates (generally of the formula R—(OCH₂CH₂)ₓ—OCH₂—COO⁻M⁺, where R is a long-chained alkyl or alkenyl group, e.g. C₃-C₂₄-alkyl or -alkenyl, x is 1 to 10 and M+ is a cation equivalent, generally Na⁺, K⁺, NH₄⁺, mono-, di- or triethanolammonium); e.g. sodium laureth carboxylate;
acylated peptides,
acyl lactylates (generally of the formula R—CO—[OCH(CH₃)—CO]ₓ—COO⁻M⁺, where R is a long-chained alkyl or alkenyl group, e.g. C₃-C₂₄-alkyl or -alkenyl, x is 3 and M+ is a cation equivalent, generally Na⁺, K⁺, NH₄⁺, mono-, di- or triethanolammonium); e.g. sodium cocoyl lactylate.

Another class of suitable anionic surfactants are polyalkoxylate polycarboxylated surfactants, e.g. as described in U.S. Pat. No. 5,376,298, EP-A-0129328, WO 03/018733 U.S. Pat. No. 5,120,326. The polyalkoxylate polycarboxylated surfactant can be described by the formula R—O—(C₂H₄O)ₓ—[CH(L)CH(L)]_{y}—[CH₂CH(CH₃)O)_{z}Q

wherein R is a hydrophobic hydrocarbon group, preferably alkyl, containing from 6 to 16, preferably from 8 to 14 carbon atoms; x is a number from 0 to 60, preferably from 4 to 50, more preferably from 6 to 50; L is either a C₁-C₃ alkyl group or a group having the formula —CH(COO⁻)—CH₂(COO⁻), with at least one L group in each molecule being —CH(COO³¹ )—CH₂(COO⁻); y is a number from 1 to 12, preferably from 2 to 10, more preferably from 3 to 8; z is a number from 0 to 20, preferably from 0 to 15, more preferably from 0 to 10; and Q is selected from the group consisting of H and sulfonate groups, the compound being rendered electrically neutral by the presence of cationic groups, preferably selected from the group consisting of sodium, potassium, and substituted ammonium, e.g. monoethanol ammonium, cations. Such polyalkoxylate polycarboxylate surfactants are commercially available under the Plurafac® brand of BASF, e.g. Plurafac® CS-10.

Cationic surfactants are, for example, ammonium salts such as C₈-C₁₆-dialkyldimethylammonium halides, dialkoxydimethylammonium halides or imidazolinium salts with a long-chain alkyl radical.

Non-ionic surfactants are typically the condensation products of one or more alkylene oxide, mostly ethylene oxide, with various reactive hydrogen-containing compounds having hydrophobic chains, for example with 8-24 carbon atoms, e.g. the condensation products of polyethyleneoxide with fatty alcohols, long chain branched alkyl alcohols, fatty acids, fatty amines, polyhydric alcohols or polypropylene oxide.

Suitable alkoxylated alcohols are listed in the following. They are of course only suitable as component (c) if they differ from the alkoxylated alkanols of component (b), i.e. if they are no C₆-C₁₄-alkanols alkoxylated with 3 to 5 units of ethylene oxide on average, e.g. if they contain more than 5 or less than 3 units of ethylene oxide on average, or alkoxylated with a total of 3 to 5 units of ethylene oxide and propylene oxide on average; if they have less than 6 or more than 14 carbon atoms in the alcohol part, if the alcohol part is not derived from an alkanol and/or if they are alternatively or additionally alkoxylated with another alkylene oxide (e.g. only with propylene oxide, PO) or alkylene diol (e.g. only with propylene-1,2- or with 1,3-diol).

Suitable alkoxylated, advantageously ethoxylated, alcohols are especially alkoxylated, advantageously ethoxylated, primary alcohols having preferably 8 to 18 carbon atoms and an average of 1 to 20, preferably 1 to 12, mol of ethylene oxide (EO) per mole of alcohol, in which the alcohol radical may be linear or branched, in particular 2-methyl-branched, or may comprise linear and methyl-branched radicals in a mixture, as are typically present in oxo alcohol radicals. Also suitable are alkyl alcohols synthesized by the Guerbet process, for example, 2-ethylhexanol, 2-n-propylheptanol, 2-isopropyl-heptanol, 2-n-butyloctanol, and 2-n-pentylnonanol, preferred are 2-ethylhexanol, 2-n-propylheptanol, and 2-isopropyl-heptanol.

More preference is given to 2-n-propylheptanol. Nonionic surfactants synthesized from this latter alcohol are marketed by BASF under the brand names Lutensol® XP and Lutensol® XL.

Other preferred ethoxylated alkyl alcohols have a higher degree of branching, especially ethoxylated alkyl alcohols available under the BASF brand names Lutensol® TO Lutensol® ON and Lutensol® TDA.

Suitable are also alcohol ethoxylates with linear radicals formed from alcohols of native origin having 12 to 18 carbon atoms, for example from coconut alcohol, palm alcohol, tallow fat alcohol or oleyl alcohol, and an average of 2 to 12 EO per mole of alcohol. The preferred ethoxylated alcohols include, for example, C₁₂-C₁₄-alcohols with 7 EO or 10 EO, C₉-C₁₁-alcohol with 7 EO or 10 EO, C₁₃-C₁₅-alcohols with 7 EO, 8 EO or 10 EO, C₁₂-C₁₃-alcohols with 7 EO or 10 EO and mixtures thereof. The degrees of ethoxylation stated are statistical averages which, for a specific product, may be an integer or a fraction. Also suitable are alcohol ethoxylates which have a narrowed homolog distribution (narrow range ethoxylates, NRE). In addition to these alkoxylated alcohols, it is also possible to use fatty alcohols with more than 12 EO. Examples thereof are tallow fat alcohol with 14 EO, 25 EO or 30 EO. It is also possible to use alkoxylated alcohols which comprise EO and PO groups together in the molecule. In this case, it is possible to use block copolymers with EO-PO block units or PO-EO block units, but also EO-PO-EO copolymers or PO-EO-PO copolymers. It will be appreciated that it is also possible to use mixed-alkoxylation nonionic surfactants in which EO and PO units are not in blockwise but in random distribution. Such products are obtainable by the simultaneous action of ethylene oxide and propylene oxide on fatty alcohols.

Suitable alkoxylated, preferably ethoxylated or ethoxylated and propoxylated, fatty acid alkyl esters preferably have 1 to 4 carbon atoms in the alkyl chain and are especially fatty acid methyl esters.

Non-ethoxylated non-ionic surfactants are for example sugar surfactants, glycerol monoethers, polyhydroxyamides (glucamide) or amine oxides.

Sugar surfactants are for example alkyl and/or alkenyl polyglycosides, sugar or alkyl sugar fatty acid esters, and fatty sugar amides.

Alkyl and/or alkenyl polyglycosides are nonionic surfactants with a carbohydrate as hydrophilic moiety and fatty alcohols or fatty acids as hydrophobic component. Examples are compounds of the formula

R—O-Gₚ,

where R is a long-chained alkyl or alkenyl group, mostly with 4-22 carbon atoms, G is an aldose or ketose moiety, mostly a glucose moiety, and p is from 1 to 10.

G is preferably derived from aldoses or ketoses having 5 or 6 carbon atoms. In one embodiment, component G is selected from the group of hexoses, preferably from the group consisting of allose, altrose, glucose, mannose, gulose, idose, galactose, talose, psicose, fructose, sorbose and tagatose, and is more preferably glucose. In another embodiment, component G is selected from the group of pentoses, preferably from the group consisting of ribulose, xylulose, ribose, arabinose, xylose and lyxose, and more preferably from xylose and arabinose.

The index number p in the above formula gives the degree of polymerization (DP), and is a number between 1 and 10. In one embodiment p is of from 1.1 to 3.0.

R can be linear or branched. For instance, the radical R is derived from linear primary alcohols, e.g. fatty alcohols, or from branched primary alcohols, in particular so-called oxo alcohols. Examples for R derived from linear primary alcohols are n-octyl, n-nonyl, n-decyl, n-undecyl, n-dodecyl, n-tridecyl, n-tetradecyl, n-pentadecly, n-heptadecyl or n-octadecyl. Examples for R derived from branched primary alcohols are isoamyl, isohexyl, isoheptyl, 2-ethylhexyl and 2-propylheptyl.

It is also possible to use mixtures of different alkyl and/or alkenyl polyglycosides Thus, all combinations of the various aldoses or ketoses with all possible alkyl- and/or alkenyl radicals can be used.

Commercially available alkyl and/or alkenyl polyglycosides are for example products sold under the PLANATAREN® and PLANTACARE® brands from Henkel, e.g. PLANTAREN 1200, PLANTAREN 1300, PLANTAREN 2000, PLANTACARE 2000, PLANTACARE 818, PLANTACARE 1200; products sold under the TRITON® CG brand from Seppic, e.g. TRITON CG 110 (or ORAMIX CG 110) and TRITON CG 312 (or ORAMIX NS 10); the product sold as LUTENSOL® GD 70 from BASF SE; the products sold under the Glucopon@brand from BASF SE, e.g. Glucopon 100 DK, Glucopon215 UP, Glucopon 225 DK, Glucopon 425 N/HH, Glucopon GD 70, Glucopon 50 G, Glucopon 600 CSUP or Glucopon 650 EC; and the product Plantatex® LLE from BASF SE.

Sugar or alkyl sugar fatty acid esters are sugar or alkyl sugar C₄-C₂₂ fatty acid esters among which there may be mentioned in particular:

(C₁-C₄)alkyl glucoside esters such as methyl glucoside monostearate, e.g. the product sold under the name GRILLOCOSE® IS by Grillowerke; methyl glucoside sesquistearate, e.g. the product sold under the name GLUCATE SS by Amerchol; 6-ethylglucoside decanoate, e.g. the product sold under the name BIOSURF 10 by Novo; the mixture of mono- and dicocoate (82/7) of 6-ethylglucoside, e.g. the product sold under the name BIOSURF® COCO by Novo; the mixture of mono- and dilaurate (84/8) of 6-ethylglucoside, e.g. the product sold under the name BIOSURF® 12 by Novo; the butyl glucoside C₁₂-C₁₈ fatty acid monoesters, such as butyl glucoside monococoate, e.g. the product sold under the names REWOPOL® V3101 or REWOSAN® V3101 and polyoxyethylenated butyl glucoside monococoate with 3 moles of ethylene oxide, e.g. the product sold under the name REWOPOL® V3122 by Rewo; glucose esters, such as 6-O-hexadecanoyl-[alpha]-D-glucose, 6-O-octanoyl-D-glucose, 6-O-oleyl-D-glucose, 6-O-linoleyl-D-glucose, which can be prepared, for example, from the corresponding acid chloride and D-glucose; sucrose monoesters such as sucrose monolaurate, e.g. the product sold under the name GRILLOTEN® LES 65, and sucrose monococoate sold under the name GRILLOTEN® LES 65K sold by the company Grillo-Werke.

The fatty sugar amides are compounds comprising at least one amide function and including at least one sugar or sugar derivative portion and at least one fatty chain; such compounds may, for example, result from the action of a fatty acid or a fatty acid derivative on the amine function of an amino sugar, or from the action of a fatty amine on a sugar comprising a carboxylic acid function (free or in lactone form) or carboxylic acid-derived function or alternatively a carbonyl function, and optionally in the presence of suitable co-reagents. Examples are N-substituted aldonamides polyhydroxylated fatty acid amides or mixtures thereof.

The N-substituted aldonamides are for example N-substituted lactobionamides, N-substituted maltobionamides, N-substituted cellobionamides, N-substituted mellibionamides and N-substituted gentiobionamides such as:
N-alkyllactobionamides, N-alkylmaltobionamides, N-alkylcellobionamides, N-alkylmellibionamides or N-alkylgentiobionamides which are mono- or disubstituted with a saturated or unsaturated, linear or branched, aliphatic hydrocarbon group which may contain heteroatoms preferably having up to 36 carbon atoms, more preferably up to 24 carbon atoms and still more particularly from 8 to 18 (for example methyl, ethyl, amyl, hexyl, heptyl, nonyl, decyl, undecyl, dodecyl, tridecyl, tetradecyl, pentadecyl, hexadecyl, heptadecyl, octadecyl; allyl, undecenyl, oleyl, linoleyl, propenyl, heptenyl), with an aromatic hydrocarbon group (for example benzyl, aniline, substituted benzyl, phenylethyl, phenoxyethyl, vinylbenzyl) or cycloaliphatic groups (for example cyclopentyl, cyclohexyl);
N-lactobionylamino acid esters where the amino acid may denote in particular: alanine, valine, glycine, lysine, leucine, arginine, aspartic acid, glutamic acid, threonine, serine, cysteine, histidine, tyrosine, methionine or which may be chosen, for example, from [beta]-alanine, sarcosine, gamma-aminobutyric acid, ornithine, citrulline or their equivalents; the said N-lactobionylamino acid esters being monosubstituted with a group of formula —(CH₂)ₙ—C(═O)—OR, where R is an aliphatic hydrocarbon group which may contain up to 36 carbon atoms and n is an integer greater than 1, as well as the corresponding N-maltobionylamino acid esters, the N-mellibionylamino acid esters, the N-cellobionylamino acid esters and the N-gentiobionylamino acid esters;
N-(alkyloxy)alkyllactobionamides which are mono- or disubstituted with a group —(CH₂)ₙ—OR′, where R′ is an aliphatic, aromatic or cycloaliphatic hydrocarbon group;
N-(polyalkyloxy)alkyllactobionamides,
N-(polyalkyloxy)alkylmaltobionamides,
N-(polyalkyloxy)alkylcellobionamides,
N-(polyalkyloxy)alkylmellibionamides or
N-(polyalkyloxy)alkylgentiobionamides which are mono- or disubstituted with a group —R′—(OR′)ₙR′R″ where R′ is an alkylene group such as ethylene, propylene or mixtures thereof, n is an integer greater than 1, R″ is a lactobionamide, maltobionamide, cellobionamide, mellibionamide or gentiobionamide group.

Examples for polyhydroxylated fatty amides are compounds of the formula

T-C(═O)—N(V)—W

where T denotes a C₅-C31 hydrocarbon group, preferably a C₇-C₁₅ linear alkyl or alkenyl chain; V denotes hydrogen, a C₁-C₄ hydrocarbon radical, 2-hydroxyethyl, 2-hydroxypropyl or mixtures thereof, preferably a C₁-C₄ alkyl such as methyl, ethyl, propyl, isopropyl, N-butyl and more particularly methyl; and W denotes a polyhydroxy hydrocarbon-containing group having a linear hydrocarbon chain with at least 3 hydroxyl groups directly attached to the chain, or an alkoxylated derivative of the said group (preferably ethoxylated or propoxylated).

W is preferably a reducing sugar derivative obtained by reductive amination reaction and more preferably a glycityl group. Glucose, maltose, lactose, galactose, mannose and xylose may be mentioned among the reducing sugars. Preferably, W is chosen from the groups of the following formulae: —(CH₂)—(CHOH)ₙ—CH₂OH; —CH—(CH₂OH)—(CHOH)ₙ₋₁—CH₂OH; and —CH₂—(CHOH)₂(CHOR′)—(CHOH)—CH₂OH, in which n is an integer ranging from 3 to 5, and R′ is hydrogen, a cyclic or aliphatic monosaccharide or one of its alkoxylated derivatives. A glycityl group in which n is 4, and in particular the group —(CH₂)—(CHOH)₄—CH₂OH, is preferred.

The group T-C(═O)—N—may be for example cocamide, stearamide, oleamide, lauramide, myristiramide, capricamide, palmitamide, tallow amide.

Non-ionic surfactants of the amine oxide type are generally of the formula R^{a}R^{b}R^{c}N⁺—O⁻, where R^{a} is a long-chained alkyl group, e.g. C₁₀-C₁₈-alkyl, preferably C₁₂-C₁₆-alkyl, and R^{b} and R^{c} are short-chained alkyl or hydroxyalkyl groups, such as methyl, ethyl or 2-hydroxyethyl. A specific example is lauryldimethylamine oxide. Moreover, the long-chained alkyl group can be derived from native sources (oils or fats), resulting in mixtures of such amine oxides, for example N-cocoalkyl-N,N-dimethylamine oxide and N-tallowalkyl-N,N-dihydroxyethylamine oxide.

Amphoteric surfactants are, for example, derivatives of secondary or tertiary amines, for example C₆-C₁₈-alkyl betaines (e.g. cocoamidopropyl betaine; disodium cocoamphodiacetate (DSCADA)) or C₆-C₁₈-alkyl sulfobetaines, or amine oxides such as alkyldimethylamine oxides.

C₂-C₃-Alkanols [component (d)] are ethanol, n-propanol and isopropanol. Mixtures thereof are also suitable.

The organic solvents different from component (d) [component (e)] generally serve for providing a stable composition, especially if the composition is a concentrate containing high amounts of organic matter.

Suitable solvents are thus polar protic or polar aprotic. Examples for suitable solvents (e) are alkanols different from C₂-C₃-alkanols, such as n-butanol or tert-butanol; C₂-C₈-alkanediols; C₁-C₈-alkylmonoethers of C₂-C₈-alkanediols; diglycols, C₁-C₈-alkylmonoethers of diglycols, polyetherpolyols; C1-C8-alkylmonoethers of polyetherpolyols; amino alcohols, such as ethanolamine, diethanolamine and triethanolamine; monophenyl ethers of C₂-C₃-alkanediols, such as 2-phenoxyethanol or phenoxypropanol.

Among the above solvents, preference is given to C₂-C₈-alkanediols and C₁-C₈-alkylmonoethers of C₂-C₈-alkanediols and to C₁-C₈-alkylmonoethers of diglycols. More preference is given to C₂-C₄-alkanediols, in particular ethylene glycol and propylene glycol, C₁-C₄-alkylmonoethers of a C₂-C₃-alkanediol, such as the C₁-C₄-alkylmonoethers of ethylene glycol or propylene glycol, specific examples being ethylene glycol monomethyl ether, ethylene glycol monoethyl ether, ethylene glycol mono-n-propyl ether, ethylene glycol mono-n-butyl ether (also termed butylglyol), propylene glycol monomethyl ether, propylene glycol monoethyl ether, propylene glycol mono-n-propyl ether, and propylene glycol mono-n-butyl ether; and to C₁-C₄-alkylmonoethers of diglycols, for example butyldiglycol, and n-hexanol ethoxylated with 1-3EO and mixtures thereof.

Sequestrants [components (f)], also termed builders, structural substances, framework substances, complexing agents, chelators, chelating agents or softeners, bind alkaline earth metals and other water-soluble metal salts without precipitating. They help to break up soil, disperse soil components, help to detach soil and in some cases themselves have a washing effect. Many of the sequestrants listed below are multi-functional, meaning that the substances have additional functions, such as a dispersing activity.

Suitable sequestrants may be either organic or inorganic in nature. Examples are aluminosilicates, carbonates, phosphates and polyphosphates, polycarboxylic acids, polycarboxylates, hydroxycarboxylic acids, phosphonic acids, e.g. hydroxyalkylphosphonic acids, phosphonates, aminopolycarboxylic acids and salts thereof, and polymeric compounds containing carboxylic acid groups and salts thereof.

Suitable inorganic sequestrants are, for example, crystalline or amorphous aluminosilicates with ion-exchanging properties, such as zeolites. Crystalline silicates suitable as sequestrants are, for example, disilicates or sheet silicates, e.g. δ—Na₂Si₂O₅ or β—Na₂Si₂O₅ (SKS 6 or SKS 7). Suitable inorganic sequestrant substances based on carbonate are carbonates and hydrogencarbonates. These can be used in the form of their alkali metal, alkaline earth metal or ammonium salts. Customary phosphates used as inorganic sequestrants are alkali metal orthophosphates and/or polyphosphates, for example pentasodium triphosphate.

Suitable organic sequestrants are, for example, C₄-C₃₀-di-, -tri- and -tetracarboxylic acids, for example succinic acid, propanetricarboxylic acid, butanetetracarboxylic acid, cyclopentanetetracarboxylic acid, and alkyl- and alkenylsuccinic acids with C₂-C₂₀-alkyl or -alkenyl radicals. Suitable organic sequestrants are also hydroxycarboxylic acids and polyhydroxycarboxylic acids (sugar acids). These include C₄-C₂₀-hydroxycarboxylic acids, for example malic acid, tartaric acid, glutonic acid, mucic acid, lactic acid, glutaric acid, citric acid, tartronic acid, glucoheptonic acid, lactobionic acid, and sucrosemono-, -di- and -tricarboxylic acid. Among these, preference is given to citric acid and salts thereof.

Another class are carboxylated fructans. Fructans are polymers of fructose molecules. They are built up of fructose residues, normally with a sucrose unit (i.e. a glucose-fructose disaccharide) at what would otherwise be the reducing terminus. The linkage of the fructose residues normally occurs at one of the two primary hydroxyls (OH-1 or OH-6). In inulin, the fructosyl residues are linked by β-2,1-linkages. In levan and phlein, the fructosyl residues are linked by β-2,6-linkages. The graminin type contains both β-2,1-linkages and β-2,6-linkages. Preferably, the carboxylated fructans are derived from inulin. Particular examples are carboxymethylinulin and carboxyethylinulin. Suitable carboxylated fructans are described in EP 3561032 A1 and WO 2010/106077.

Suitable organic sequestrants are also phosphonic acids, for example hydroxyalkylphosphonic acids or aminophosphonic acids, and the salts thereof. These include, for example, phosphonobutanetricarboxylic acid (2-phosphinobutane-1,2,4-tricarboxylic acid; PBTC), aminotris-methylenephosphonic acid (N[CH₂PO(OH)₂]₃), aminotris(methylenephosphonate), sodium salt (ATMP; N[CH₂PO(ONa)₂]₃), ethylenediaminetetra(methylenephosphonic acid) (EDTMPA), hexamethylenediamine(tetra-methylenephosphonic acid), hexamethylenediamine(tetramethylenephosphonate), potassium salt (C₁₀H₍₂₈₋ₓ₎N₂KₓO₁₂P₄(x=6)), bis(hexamethylene)triamine(pentamethylenephosphonic acid) ((HO₂)POCH₂N[(CH₂)₂N[CH₂PO(OH)₂]₂]₂), diethylenetriamine-penta(methylene-phosphonic acid) (DTPMP; (HO)₂POCH₂N[CH₂CH₂N[CH₂PO(OH)₂]₂]₂), diethylenetriaminepenta(methylenephosphonate), sodium salt (C₉H₍₂₈₋ₓ₎N₃NaₓO₁₅P₅(x=7)); tetramethylene-triamine-pentaphosphonic acid, hydroxyethylamine diphosphonic acid, 2-hydroxyethyliminobis(methylenephosphonic acid) (HOCH₂CH₂N[CH₂PO(OH)₂]₂), morpholinomethanediphosphonic acid, 1-hydroxy-C₁- to C₁₀-alkyl-1,1-diphosphonic acids such as 1-hydroxyethane-1,1-diphosphonic acid (HEDP; CH₂C(OH)[PO(OH)₂]₂). Suitable organic sequestrants are moreover polyasparatic acids. Polyaspartic acid include salts of polyaspartic acids. Salt forming cations may be monovalent or multivalent, examples being sodium, potassium, magnesium, calcium, ammonium, and the ammonium salt of mono-, di- and triethanolamine. Such polymers may be co-polymers, in particular of (a) L- or D-aspartic acid (preferably L-aspartic acid), (b) a carboxylic acid and (c) a diamone or an amino alcohol. Such copolymers generally comprise 70-95 mol % of (a), 5-30 mol % of (b) and 2-20 mol % of (c). The molar ratio of the carboxyl-containing compound (b) to the diamine or amino alcohol (c) is preferably between 5:1 and 1:1.5 or between 3:1 and 1:1.2, and more preferably between 3:1 and 1:1 or 2:1 and 1:1. Suitable organic sequestrants are additionally aminopolycarboxylic acids, such as nitrilotriacetic acid (NTA), nitrilomonoacetic dipropionic acid, nitrilotripropionic acid, β-alaninediacetic acid (β-ADA), ethylenediaminetetraacetic acid (EDTA), diethylenetriaminepentaacetic acid (DTPA), 1,3-propylenediaminetetraacetic acid, 1,2-propylenediaminetetraacetic acid, N-(alkyl)ethylenediaminetriacetic acid, N-(hydroxyalkyl)ethylenediaminetriacetic acid, ethylenediaminetriacetic acid, cyclohexylene-1,2-diaminetetraacetic acid, iminodisuccinic acid, ethylenediaminedisuccinic acid, serinediacetic acid, isoserinediacetic acid, L-asparaginediacetic acid, L-glutaminediacetic acid, methylglycinediacetic acid (MGDA), and the salts of the aforementioned aminopolycarboxylic acids. Suitable organic sequestrants are additionally polymeric compounds containing carboxylic acid groups, such as acrylic acid homopolymers. The term “acrylic acid homopolymer” also comprises polymers in which some or all of the carboxylic acid groups are present in neutralized form. Suitable polymeric compounds containing carboxylic acid groups are also oligomaleic acids. Suitable polymeric compounds containing carboxylic acid groups are also terpolymers of unsaturated C₄-C₈-dicarboxylic acids. Suitable unsaturated C₄-C₈-dicarboxylic acids in this context are, for example, maleic acid (or maleic anhydride), fumaric acid, itaconic acid, aconitic acid, mesaconic acid, methylenemalonic acid and citraconic acid. Suitable polymeric compounds containing carboxylic acid groups are also homopolymers of the monoethylenically unsaturated C₃-C₈-monocarboxylic acids, for example acrylic acid, methacrylic acid, crotonic acid, 2-ethylacrylic acid, 2-phenylacrylic acid, cinnamic acid, vinylacetic acid and sorbic acid, copolymers of dicarboxylic acids, for example of maleic acid and acrylic acid; terpolymers of maleic acid, acrylic acid and a vinyl ester of a C₁-C₃-carboxylic acid; and copolymers of maleic acid with C₂-C₈-olefins.

Further additives [component (g)] are for example pH adjusting agents (pH modifiers), thickeners, antifreezing agents, antifoaming agents, colorants and perfumes [i.e. different from components (a) and (b)].

Depending on the desired pH of the composition, pH adjusting agents (pH modifiers) are acids or bases. The pH can also be adjusted by buffering systems.

The acids can be inorganic or organic. Suitable inorganic acids are for example sulfuric acid, hydrochloric acid and phosphoric acid, where sulfuric acid is generally preferred. Suitable organic acids are for example aliphatic, saturated non-substituted C₁-C₆-mono-, di- and tri-carboxylic acids such as formic acid, acetic acid, propanoic acid, oxalic acid, succinic acid, glutaric acid and adipic acid; aliphatic, saturated C₁-C₆-mono-, di- and tri-carboxylic acids carrying one or more OH groups, such as glycolic acid, lactic acid, tartric acid and citric acid; aliphatic, unsaturated C₁-C₆-mono-, di- and tri-carboxylic acids such as sorbic acid; aromatic carboxylic acids, such as benzoic acid, salicylic acid and mandelic acid; and sulfonic acids, such as methanesulfonic acid or toluenesulfonic acid. The organic acids mainly serve for adapting the pH of the composition, but some of them, e.g. the di- and tricarboxylic acids, can also act as sequestrants.

Suitable bases are in particular inorganic bases, such as the carbonates mentioned in context with the sequestrant, e.g. sodium or potassium carbonate; further ammonium carbonate, alkali metal and earth alkaline metal bicarbonates, such as sodium hydrogencarbonate or potassium hydrogencarbonate, alkali metal and earth alkaline metal hydroxides, such as NaOH or KOH, or ammonium hydroxide. Organic bases can also be used; examples are alkanolamines, such as monoethanolamine, triethanolamine or aminomethylpropanol, or guanidine derivatives, such as 1,1,3,3-tetramethylguanidine or triazabicyclodecene.

Suitable buffering agents are the typical systems, such as hydrogenphosphate/dihydrogenphosphate buffer, carbonate/hydrogencarbonate buffer, acetic acid/acetate buffer or Tris buffer. Moreover, most of the above acids which are weak and the anion of which is not a strong salt also have buffering capacity.

The thickeners serve to impart the desired viscosity to the composition of the invention.

Any known thickener (rheology modifier) is suitable in principle, provided that it does not exert any adverse effect on the efficacy of the composition. Suitable thickeners may either be of natural origin or of synthetic nature.

Thickeners of natural origin are mostly derived from polysaccharides. Examples are xanthan, gellan gum, carob flour, guar flour or gum, carrageenan, agar, tragacanth, gum arabic, alginates, modified starches such as hydroxyethyl starch, starch phosphate esters or starch acetates, dextrins, pectins and cellulose derivatives, such as carboxymethylcellulose, hydroxyethylcellulose, hydrophobically modified hydroxyethyl cellulose, hydroxypropylcellulose, hydroxypropylmethylcellulose, methylcellulose and the like. Thickeners of natural origin are also inorganic thickeners, such as polysilicic acids and clay minerals, for example sheet silicates, and also the silicates mentioned for the builders.

Examples of synthetic thickeners are polyacrylic and polymethacrylic compounds, such as (partly) crosslinked homopolymers of acrylic acid, for example homopolymers of acrylic acid which have been crosslinked with an allyl ether of sucrose or pentaerythritol, or with propylene (carbomers), for example the Carbopol® brands from BF Goodrich (e.g. Carbopol® 676, 940, 941, 934 and the like) or the Polygel® brands from 3V Sigma (e.g. Polygel® DA), copolymers of ethylenically unsaturated mono- or dicarboxylic acids, for example terpolymers of acrylic acid, methacrylic acid or maleic acid with methyl acrylate or ethyl acrylate and a (meth)acrylate which derives from long-chain ethoxylated alcohols, for example the Acusol® brands from Rohm & Haas (e.g. Acusol® 820 or 1206A), copolymers of two or more monomers which are selected from acrylic acid, methacrylic acid and the C₁-C₄-alkyl esters thereof, for example copolymers of methacrylic acid, butyl acrylate and methyl methacrylate or of butyl acrylate and methyl methacrylate, for example the Aculyn® and Acusol® brands from Rohm & Haas (e.g. Aculyn® 22, 28 or 33 and Acusol® 810, 823 and 830), or crosslinked high molecular weight acrylic acid copolymers, for example copolymers of C₁₀-C₃₀-alkyl acrylates with one or more comonomers selected from acrylic acid, methacrylic acid and the C₁-C₄-alkyl esters thereof, said copolymers having been crosslinked with an allyl ether of sucrose or pentaerythritol (e.g. Carbopol® ETD 2623, Carbopol® 1382 or Carbopol® AQUA 30 from Rohm & Haas). Another preferred substance group is the Rheovis® brands from BASF, e.g. Rheovis® AT 120.

Examples for suitable antifreezing agents are ethylene glycol, propylene glycol, urea and glycerine.

Examples for suitable antifoaming agents are silicones, long-chain alcohols and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Fragrances can be of natural or synthetic origin; their nature is in general not critical.

The Bacillus strains of the invention may be produced via fermentation. As used herein, the term “fermentation” refers generally to any activity or process involving the decomposition (digestion) of organic materials by the Bacillus strain of the invention resulting in its growth. The term fermentation as used herein is to be understood to include any type of fermentation including but not limited to substantially zero growth fermentation, i.e. a fermentation process wherein the cell density of the microorganisms applied in the fermentation process will be substantially zero and wherein the microorganisms will not undergo a significant population growth, including under-fermentation, i.e. a fermentation process wherein the fermentation is performed during an insufficient period of time, and including over-fermentation, e.g. a fermentation process wherein fermentation is performed for a too long period of time. The term “fermentation” encompasses both anaerobic and aerobic processes, as well as processes involving a combination or succession of one or more anaerobic and/or aerobic stages. Furthermore, the fermentation may be a continuous or a batch process.

There are two determining factors that trigger sporulation: nutrient starvation and cell density. Thus, in order to produce spores of the Bacillus strain of the invention, the vegetative bacterial cells may be exposed to nutrient starvation in combination with a high cell density, such as an OD₆₀₀ greater than 0.3, greater than 0.4, greater than 0.5 or greater than 0.6. Alternatively, the cell density may be greater than 10⁶ cells/mL, greater than 10⁷ cells/mL, 10⁸ cells/mL, 5*10⁸ cells/mL or 10⁹ cells/mL.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all of these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of members, this is meant to also encompass a group which consists of these members only.

The present invention is further illustrated by the following examples, sequences and figures from which further features, embodiments, aspects and advantages of the present invention may be taken. Further, the materials, methods, and examples are illustrative only and are not intended to be limiting, unless otherwise specified.

### EXAMPLES

Each strain of a collection comprising around 50 strains from a variety of different bacterial species has been tested for its ability to form spores, to provide extracellular enzyme activity and to reduce malodor according to the below protocols. A score for each criterium has been allocated and the strains with the highest total score have further been tested for cleaning effects on dirty tiles and long-term spore viability.

### Example 1—Spore Count

Before use, all strains were streaked on TSAY-agar plates (30 g/L Tryptic Soy Broth, 3 g/L yeast extract, 20 g/L agar) and incubated at 30 °C for 2 days. All sporulation experiments were performed in BA055 medium containing 10 g/L Glucose*H₂O, 5 g/L yeast extract, 10 g/L Sofarin Soymeal, 10 g/L corn starch, 3.7 g/L K₂HPO₄ and 2.5 g/L KH₂PO₄ adjusted to pH 7 and sterilized at 121 °C for 30 min. To determine the ability of bacterial cultures to produce viable spores, spore count measurements were performed by inoculating 1 mL BA055 medium in flower plates (m2p-labs) with a single colony. Plates were incubated at 30 °C, 1000 rpm for 72 h. Afterwards, dilution series were performed by mixing 100 μL sample, with 900 μL dilution solution containing 0.9% NaCl and 0.1 g/L Tween80 and mixed by vortexing for 30 s. Dilutions were repeated until adequate dilution. 10 μL of diluted samples were spotted on agar plates, incubated for 24 h at 30 °C and cells counted. To differentiate vegetative cells from spores, 100 μL of diluted cell suspensions were transferred to a 96-well plate and incubated in a thermocycler at 60 °C for 30 min. 10 μL of heat-treated samples were spotted on NB-agar plates and incubated for 24 h at 30 °C before cells were counted. For a semi-quantitative rating, 1*10⁸ spores/mL were rated 1, 2*10⁸ spores/mL were rated 2, 3*10⁸ spores/mL were rated 3 and continuing with increasing spore count. Strains that reached a spore count >10⁹ spores/mL after 72 h were rated 10, while strains <10⁸ spores/mL were rated 0.

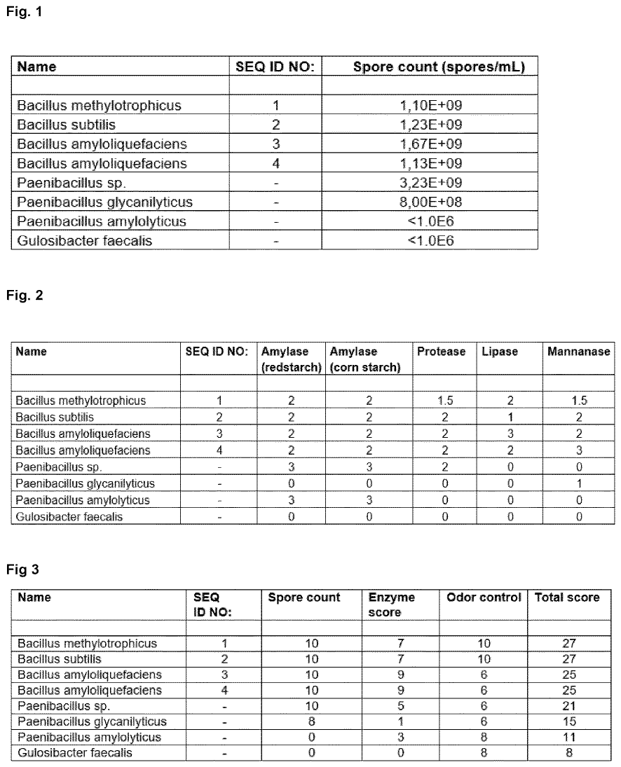

FIG. 1 shows the spore count of four bacterial strains, namely Bacillus methylotrophicus (comprising SEQ ID NO:1), Bacillus subtilis (comprising SEQ ID NO:2), Bacillus amyloliquefaciens (comprising SEQ ID NO:3) and Bacillus amyloliquefaciens (comprising SEQ ID NO:4) which had the highest total score of all bacterial strains tested. As comparative examples data for four additional strains, namely Paenibacillus sp., Paenibacillus glycanilyticus, Paenibacillus amylolyticus and Gulosibacter faecalis is shown. The spore count score achieved by each strain is shown in FIG. 3 in the column “spore count”.

### Example 2—Agar Plate Enzyme Assays

For agar plate enzyme assays, strains were streaked on NB-agar based plates (8 g/L NB (Difco), 20 g/L agar) containing further additives to allow semiquantitative visual evaluation of enzymatic activities. To assess amylase activity, plates additionally contained 2.5 g/L red starch. In a second approach, agar plates contained 5 g/L soluble corn starch and were stained with Lugolsch solution after 48 h incubation. For protease activity, NB-agar plates additionally contained 10 g/L skim milk powder. To assess lipase activity, NB-agar was mixed with 10 ml/L tributyrin before pouring the agar plates. For mannanase activity, 1 g/L Locust Bean Gum was added to the NB-agar. After 48 h of incubation on plates, the respective enzyme activities were assessed visually. High enzymatic activities resulted in large, clear halo rings around bacterial colonies due to degradation of the respective substrates. Enzyme activities were scored semi-quantitatively from 0 (no activity) to 3 (high activity). The values of the two different assay concerning amylase activity were added and the sum was divided by two to receive an average amylase activity.

FIG. 2 shows the score for the in Example 1 described strains for each enzymatic assay tested. A total enzyme score was generated by adding the scores for the average amylase, protease, lipase and mannanase activity. This score is shown in the column “enzyme score” in FIG. 3.

### Example 3—Odor Control

Strains were cultivated in 1 mL BA055 medium in flower plates (m2p-lab) at 30 °C, 1000 rpm. Biomass was separated by centrifugation (4,500 g, 10 min) and supernatant discarded. Flower plates were sealed again with an AirPore lid and dried at RT. After 7 days, pellets containing approximately 1*10⁹ viable spores were resuspended in 1 mL lactose-free whole milk (not sterilized, lactose free, 3.5% fat) and transferred to a sterile 50 mL falcon tube, respectively. Tubes were incubated at 30 °C for one week without agitation. Odor formation was evaluated olfactorily by sniffing (10=no odor/milk odor, 8=slightly acidic odor, 6=significantly acidic, slight malodor, 4=moderate malodor, 2=strong malodor).

The respective scores for the different strains (Bacillus methylotrophicus (comprising SEQ ID NO:1), Bacillus subtilis (comprising SEQ ID NO:2), Bacillus amyloliquefaciens (comprising SEQ ID NO:3), Bacillus amyloliquefaciens (comprising SEQ ID NO:4), Paenibacillus sp., Paenibacillus glycanilyticus, Paenibacillus amylolyticus and Gulosibacter faecalis) are shown in FIG. 3, column “odor control”.

Based on the total score, the strains Bacillus methylotrophicus (comprising SEQ ID NO:1), Bacillus subtilis (comprising SEQ ID NO:2), Bacillus amyloliquefaciens (comprising SEQ ID NO:3) and Bacillus amyloliquefaciens (comprising SEQ ID NO:4) were selected for further tests.

### Example 4—Long-Term Cleaning Effects (CFT Tiles)

20 mL of BA055 medium in 100 mL baffled shaking flasks were inoculated with the target strains and incubated at 30 °C 220 rpm for 72 h. Spores were harvested by centrifugation and washed with dH₂O and resuspended in either dH₂O or a sterile filtered surfactant based on 1×Novozymes Deep Clean Multi EU 5× EP. 4 mL solution containing at least 3*10⁸ cfu/mL were spread on different types of CFT tiles to assess long term cleaning effects on different substrates(DM-40: Hardsurface Cleaner soil based on IKW protocol; DM-177: Mix corn/starch, coloured, extra severe on melamin; DM-03: shepard's pie on melamin). All CFT tiles were placed in Corning agar plates and incubated at 30 °C for 7 days. Afterwards, plates were rinsed with dH₂O, dried and visually or photodensitometrically evaluated using a Mach5 device.

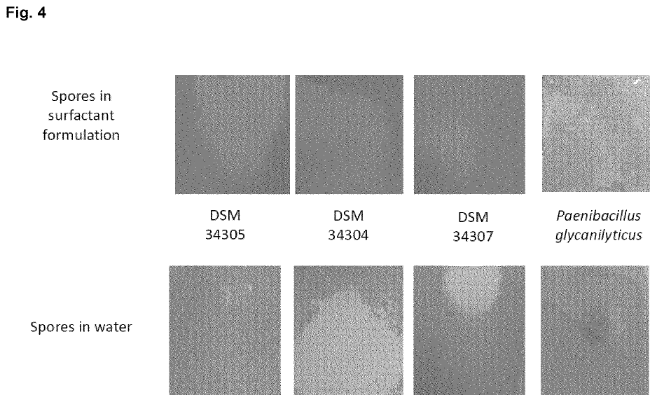

The effects of long-term cleaning for Bacillus subtilis (DSM 34307), Bacillus amyloliquefaciens (DSM 34304) as well as Bacillus amyloliquefaciens (DSM 34305) can be seen in FIG. 4. Said figure depicts the cleaning effects for the indicated bacterial strains on tiles soiled with shepherd's pie (Paenibacillus glycanilyticus is an additionally tested strain of the library that shows no effect in long-term cleaning). Significant cleaning effects were observed for all four bacterial strains on DM-03 CFT tiles soiled with a complex medium (shepherd's pie) containing a mixture of proteins, starch as well as fat.

### Example 5—Long Term Spore Viability

Long term spore viability was assessed by inoculating 50 mL of BA055 medium with bacterial strains and incubation at 30 °C, 200 rpm. After 5 days of incubation, biomass was harvested by centrifugation, washed three times with 50 mL dH₂O, followed by resuspension in 60% glycerol. pH of glycerol solutions was adjusted to pH 5/7/9 respectively using 0.1 M NaOH or 0.1 M HCl. Spore containing glycerol solutions were stored in 15 mL plastic tubes at RT. Spore count was measured in 7 day intervals as described above.

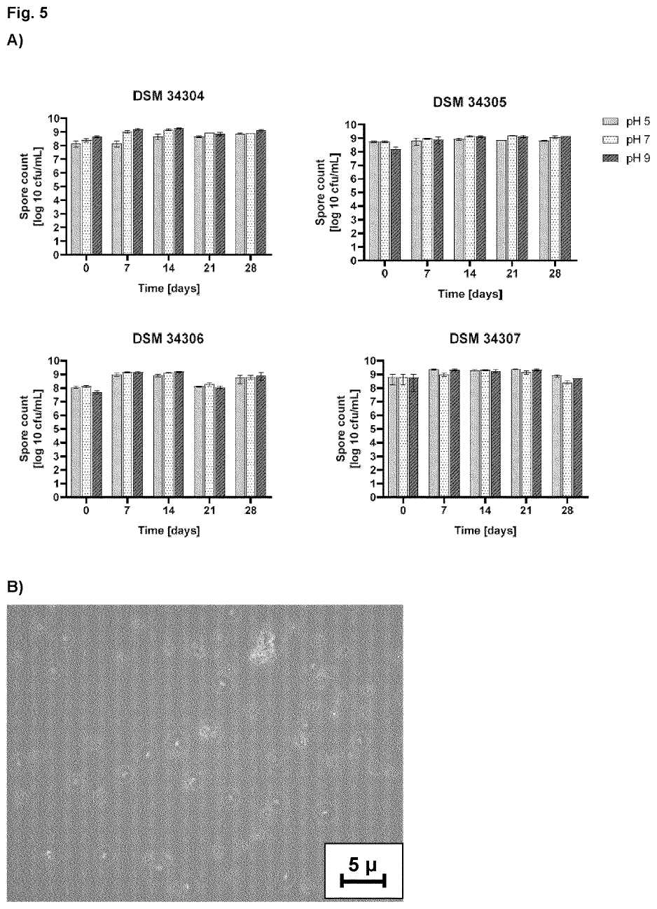

For all four selected test strains, the spore count remained stable over a period of 4 weeks, indicating high stability of the strains in the selected pH range (FIG. 5 A). Furthermore, no germination of the spores was observed microscopically as long as the spores remained in the glycerol solution. FIG. 5 B shows bright-field microscopy of Bacillus amyloliquefaciens (comprising SEQ ID NO:3) stored at pH 9 after 14 days (100× magnification) demonstrating the sole presence of spores without any vegetative cells.

### Example 6: Comparison of Inventive Strains with Reference Strains Via Sensory Malodor Determination and Via HPLC Measurement

1 mL of lactose-free milk (3.5% fat) was inoculated with 1*10\hat{ }9 spores/mL in a 15 mL falcon tube and incubated at 30 °C for 7 days. Afterwards, odor formation was evaluated by a sensory panel (0=strong malodor formation, 10=no odor formation; see also Example 3). After sensory evaluation, samples were diluted with 1 mL dH2O, mixed by pipetting and centrifuged (6,000 g, 10 min). The supernatant was filtered using a 0.22 μm PES filter and transferred to a glass vial and measured via HPLC (Aminex HPX-87-H (BIO-RAD), 300×7.8 mm, 9 μm particle size, with 0.5 M H2SO4 (Roth) as eluent and 0.5 ml/min flow rate at 30 °C).

The correlation of microbial caused malodor with the presence of certain metabolite compounds is well-known in the art. Along this line Greenman et al. (Greenman, John et al. Journal of the American Dental Association (1939), 136,6 (2005): 749-57) conceived an organoleptic model that correlates the sensory evaluation of a given sample with the concentration of different compounds. For example, a commonly found malodor compound is butyric acid, which shows a strong malodor perception at relatively low concentrations in its protonated form. Already low concentrations >0.8 g/L cause a strong odor perception. The sensory malodor determinations for the inventive strains as well as the control and reference strains correlate well with the proposed butyric acid concentrations and olfactory perceptions as described by Greenman et al. (see FIG. 6). B. subtilis ATCC 6633, which was previously described as a suitable strain for probiotic cleaning applications (Vehapi, M., Özgimen, D. Bulletin of Biotechnology 1 (2020): 1-7), showed a significantly bad odor profile, both concerning the formation of butyric acid in the milk samples (0.44 g/L) as well as sensory perception by the panel. An additional publicly available Bacillus amyloliquefaciens strain (DSM 1060) demonstrated bad odor control behavior as well. Contrarily, milk samples inoculated with the inventive strains (containing SEQ ID Nos. 1-4) demonstrated an overall positive odor profile, which was analytically confirmed by the composition of organic acids in the samples. Accordingly, milk samples inoculated with these strains did not show any or only low concentrations of butyric acid (max. 0.2 g/L) resulting in no detectable or only slight odor and an overall positive organoleptic perception by the test panel.

### Example 7—ADW Formulations

In the following there are exemplified ADW (automatic dishwasher) formulations that contain the inventive strains of the present application:

Automatic Dishwasher Detergents

| | A (wt %) | B (wt %) | C (wt %) |
|---|---|---|---|
| Sodium Carbonate | 5-10 | | 30-40 |
| Sodium Bicarbonate | | 15-25 | |
| Sodium Silicate | 10-12 | 1-3 | 0.5-2 |
| MGDA | 1-7 | | 1-7 |
| GLDA | | 1-7 | |
| Nonionic surfactant¹ | | 1-2 | 0.5-2 |
| Polymer dispersant² | 3-5 | | |
| Polymer with Zinc Ion³ | 0.5-2 | 1-3 | 1-5 |
| Enzymes | | 0-1 | 0-1 |
| Bleach, bleach catalyst, | 0.5-2 | | |
| bleach activitor | | | |
| Sodium Hypochloride | 0.5-1.5 | | |
| Sodium Sulfate | 2-5 | 2-5 | 20-60 |
| Sodium Benzoate | 0.5-1.5 | 0.5-1.5 | |
| Perfume | 0.01-0.1 | 0.01-0.1 | 0.01-0.1 |
| Xanthan gum | | 0.5-1.5 | |
| Polygel DKP | 1-2 | | |
| Inventive bacterial strain | 0.001-5 | 0.001-5 | 0.001-5 |
| Water, dye and other | Balance to | Balance to | Balance to |
| adjuncts | 100 wt % | 100 wt % | 100 wt % |

¹SLF-18 POLY TERGENT from the BASF Corporation
²Copolymer ACUSOL ® 425N from Dow
³Polymer with Zinc Counterion

Automatic Dishwashing Unit-Dose Products (pouches)

| Particulate composition | wt % |
|---|---|
| STPP | 0 |
| Silicate | 1-5 |
| Sodium Carbonate | 25-50 |
| MGDA | 5-25 |
| Polymers with Zinc Ion¹ | 5-10 |
| Polymer Dispersant² | 0-5 |
| Nonionic Surfactant³ | 3-10 |
| Enzyme | 0-6 |
| Bleach, Bleach Catalyst, Activator | 5-15 |
| Perfume | 0.05-0.2 |
| Sodium Sulfate | 0-20 |
| Inventive bacterial strain | 0.001-5 |
| Liquid composition | |
| DPG | 40-50 |
| Nonionic Surfactant³ | 40-50 |
| Neodol C11E9 | 0-5.0 |
| Glycerine | 0-5.0 |
| Dye | 0.1-1.0 |

¹A copolymer or any mixture of copolymers with zinc ions defined in the Polymer Section
²ACUSOL ® 445N from Dow
³SLF-18 POLY TERGENT from the BASF Corporation

### Example 8—Cleaning Formulations

In the following there are exemplified cleaning formulations that contain the inventive strains of the present application:

Strong acidic sanitary cleaner based on
lactic acid/methanesulfonic acid (%)

| | A | B | C |
|---|---|---|---|
| | (wt %) | (wt %) | (wt %) |
| Lactic acid | 7.1 | 7.1 | 7.1 |
| Methanesulfonic acid | 2.8 | 2.8 | 2.8 |
| Sodium octylsulfat salt (anionic tenside) | — | — | 0.95 |
| Fatty alcohol C10-6 EO (non-ionic tenside) | — | 0.95 | — |
| Perfume | — | 0.15 | 0.15 |
| Fatty alcohol C10-6 EO (perfume emulsifier) | — | — | 0.1 |
| Dye (red) | 0.001 | 0.001 | 0.001 |
| Inventive bacterial strain | 0.001-5 | 0.001-5 | 0.001-5 |
| Water | ad 100 | ad 100 | ad 100 |
| pH value (20 °C) | 0.6 | 0.6 | 0.6 |

The following I-VIII are ready-to-use (RTU) cleaner formulations (i.e., formulations that are not further diluted with water before use) which contains the Bacillus spores of the invention. Such formulations provide a good initial cleaning effect. The spores are fully stable in the formulation and upon use, spore germination is not inhibited. Such formulations can be used to treat general hard surfaces in the home or in offices or in commercial-, industrial-, or agricultural settings. But especially for surfaces in bathrooms, kitchens, dairies, and other places where food is stored or treated.

| Component name | I | II | III | IV | V | VI | VII | VIII |
|---|---|---|---|---|---|---|---|---|
| Endospores of | 10⁴-10⁹ | 10⁴-10⁹ | 10⁴-10⁹ | 10⁴-10⁹ | 10⁴-10⁹ | 10⁴-10⁹ | 10⁴-10⁹ | 10⁴-10⁹ |
| Bacillus | cfu/ml | cfu/ml | cfu/ml | cfu/ml | cfu/ml | cfu/ml | cfu/ml | cfu/ml |
| methylotrophicus | | | | | | | | |
| (DSM 34306) | | | | | | | | |
| C13 Linear alkyl | 1 | 1 | | | | | 1 | |
| benzene sulfonate | | | | | | | | |
| sodium salt | | | | | | | | |
| Laurylethersulphate | | | 1 | 2 | | | | |
| (2EO) -Na salt | | | | | | | | |
| Lutensol XP 70 | 1 | | 1 | | 0.5 | | | 0.2 |
| (C₁₀ Guerbet | | | | | | | | |
| alcohol + 7 EO) | | | | | | | | |
| Lutensol A7N | | 0.5 | | | | 0.5 | | |
| (Linear C₁₂-C₁₄ | | | | | | | | |
| alcohol + 7EO) | | | | | | | | |
| Cocoamidopropylbetaine | | | | 0.5 | | | | |
| Laurylamine oxide | | | | | | | | 1 |
| C₈-C₁₀ | | | | | 2 | | | |
| alkylpolyglycoside | | | | | | | | |
| C₁₂-C₁₄ | | | | | | 2 | | |
| alkylpolyglycoside | | | | | | | | |
| Sodium Palm | | | | | | | 0.5 | |
| kernelate (soap) | | | | | | | | |
| Na₂CO₃/NaHCO₃ | 0-2 | 0-2 | 0-2 | 0-2 | 0-2 | 0-2 | 0-2 | 0-2 |
| Monoethanolamine | 0-2 | 0-2 | 0-2 | 0-2 | 0-2 | 0-2 | 0-2 | 0-2 |
| Butyldiglycol | 0-2 | 0-2 | 0-2 | 0-2 | 0-2 | 0-2 | 0-2 | 0-2 |
| Ethanol | 0-2 | 0-2 | 0-2 | 0-2 | 0-2 | 0-2 | 0-2 | 0-2 |
| propyleneglycol | 0-2 | 0-2 | 0-2 | 0-2 | 0-2 | 0-2 | 0-2 | 0-2 |
| MGDA/Trilon M | 0-1 | 0-1 | 0-1 | 0-1 | 0-1 | 0-1 | 0-1 | 0-1 |
| (active Na₃-salt) | | | | | | | | |
| Sodium citrate | 0-1 | 0-1 | 0-1 | 0-1 | 0-1 | 0-1 | 0-1 | 0-1 |
| Sodium | 0-0.5 | 0-0.5 | 0-0.5 | 0-0.5 | 0-0.5 | 0-0.5 | 0-0.5 | 0-0.5 |
| diethylenetriamine | | | | | | | | |
| pentamethylene | | | | | | | | |
| phosphonate | | | | | | | | |
| 2-phenoxyethanol | 0-2 | 0-2 | 0-2 | 0-2 | 0-2 | 0-2 | 0-2 | 0-2 |
| Benzisothiazolinone | 0-0.01 | 0-0.01 | 0-0.01 | 0-0.01 | 0-0.01 | 0-0.01 | 0-0.01 | 0-0.01 |
| Methylisothazolinone | 0-0.005 | 0-0.005 | 0-0.005 | 0-0.005 | 0-0.005 | 0-0.005 | 0-0.005 | 0-0.005 |
| bronopol | 0-0.05 | 0-0.05 | 0-0.05 | 0-0.05 | 0-0.05 | 0-0.05 | 0-0.05 | 0-0.05 |
| Benzoic acid/Na- | 0-1 | 0-1 | 0-1 | 0-1 | 0-1 | 0-1 | 0-1 | 0-1 |
| benzoate | | | | | | | | |
| Benzylalcohol | 0-1 | 0-1 | 0-1 | 0-1 | 0-1 | 0-1 | 0-1 | 0-1 |
| Formic acid/Na- | 0-1 | 0-1 | 0-1 | 0-1 | 0-1 | 0-1 | 0-1 | 0-1 |
| Formate | | | | | | | | |
| Sorbic acid/K- | 0-1 | 0-1 | 0-1 | 0-1 | 0-1 | 0-1 | 0-1 | 0-1 |
| Sorbate | | | | | | | | |
| Perfumes/dyes/ | yes | yes | yes | yes | yes | yes | yes | yes |
| others | | | | | | | | |
| Hydrochloric acid | 4-8 | 4-8 | 4-8 | 4-8 | 4-8 | 4-8 | 4-8 | 4-8 |
| or sodium | | | | | | | | |
| hydroxide to reach | | | | | | | | |
| pH | | | | | | | | |
| Deionized water | Up to | Up to | Up to | Up to | Up to | Up to | Up to | Up to |
| | 100% | 100% | 100% | 100% | 100% | 100% | 100% | 100% |

The formulations I-a to VIII-a correspond to the same formulations as I to VIII, but with Bacillus subtilis (DSM 34307) instead of Bacillus methylotrophicus (DSM 34306).

The formulations I-b to VIII-b correspond to the same formulations as I to VIII, but with Bacillus amyloliquefaciens (DSM 34304) instead of Bacillus methylotrophicus (DSM 34306). The formulations I-c to VIII-c correspond to the same formulations as I to VIII, but with Bacillus amyloliquefaciens (DSM 34305) instead of Bacillus methylotrophicus (DSM 34306).

The following are dilutable, concentrated cleaning formulations (i.e., formulations that are diluted 10-100× with water before use) which contains XYZ spores of the invention. The spores are fully stable in the formulation. After dilution with water, such formulations provide a good initial cleaning effect, and upon use, spore germination is not inhibited. Such formulations can be used to treat general hard surfaces in the home or in offices or in commercial-, industrial-, or agricultural settings. But especially for surfaces in bathrooms, kitchens, dairies, and other places where food is stored or treated

| Component name | IX | X | XI | XII | XIII | XIV | XV | XVI |
|---|---|---|---|---|---|---|---|---|
| Endospores of | 10⁴-10⁹ | 10⁴-10⁹ | 10⁴-10⁹ | 10⁴-10⁹ | 10⁴-10⁹ | 10⁴-10⁹ | 10⁴-10⁹ | 10⁴-10⁹ |
| Bacillus | cfu/ml | cfu/ml | cfu/ml | cfu/ml | cfu/ml | cfu/ml | cfu/ml | cfu/ml |
| methylotrophicus | | | | | | | | |
| (DSM 34306) | | | | | | | | |
| C13 Linear alkyl | 4 | 3 | | | | | 4 | |
| benzene sulfonate | | | | | | | | |
| sodium salt | | | | | | | | |
| Laurylethersulphate | | | 3 | 4 | | | | |
| (2EO)-Na salt | | | | | | | | |
| Lutensol XP 70 (C₁₀ | 1 | | 2 | | 2 | | | 2 |
| Guerbet alcohol + 7 | | | | | | | | |
| EO) | | | | | | | | |
| Lutensol A7N | | 1 | | | | 2 | | |
| (Linear C₁₂-C₁₄ | | | | | | | | |
| alcohol + 7EO) | | | | | | | | |
| Cocoamidopropylbetaine | | | | 1 | | | | |
| Laurylamine oxide | | | | | | | | 4 |
| C₈-C₁₀ | | | | | 4 | | | |
| alkylpolyglycoside | | | | | | | | |
| C₁₂-C₁₄ | | | | | | 5 | | |
| alkylpolyglycoside | | | | | | | | |
| Sodium Palm | | | | | | | 2 | |
| kernelate (soap) | | | | | | | | |
| Na₂CO₃/NaHCO₃ | 0-3 | 0-3 | 0-3 | 0-3 | 0-3 | 0-3 | 0-3 | 0-3 |
| Monoethanolamine | 0-3 | 0-3 | 0-3 | 0-3 | 0-3 | 0-3 | 0-3 | 0-3 |
| Butyldiglycol | 0-4 | 0-4 | 0-4 | 0-4 | 0-4 | 0-4 | 0-4 | 0-4 |
| Ethanol | 0-4 | 0-4 | 0-4 | 0-4 | 0-4 | 0-4 | 0-4 | 0-4 |
| propyleneglycol | 0-4 | 0-4 | 0-4 | 0-4 | 0-4 | 0-4 | 0-4 | 0-4 |
| MGDA/Trilon M | 0-3 | 0-3 | 0-3 | 0-3 | 0-3 | 0-3 | 0-3 | 0-3 |
| (active Na₃-salt) | | | | | | | | |
| Sodium citrate | 0-4 | 0-4 | 0-4 | 0-4 | 0-4 | 0-4 | 0-4 | 0-4 |
| Sodium | 0-1 | 0-1 | 0-1 | 0-1 | 0-1 | 0-1 | 0-1 | 0-1 |
| diethylenetriamine | | | | | | | | |
| pentamethylene | | | | | | | | |
| phosphonate | | | | | | | | |
| 2-phenoxyethanol | 0-2 | 0-2 | 0-2 | 0-2 | 0-2 | 0-2 | 0-2 | 0-2 |
| Benzisothiazolinone | 0-0.01 | 0-0.01 | 0-0.01 | 0-0.01 | 0-0.01 | 0-0.01 | 0-0.01 | 0-0.01 |
| Methylisothazolinone | 0-0.005 | 0-0.005 | 0-0.005 | 0-0.005 | 0-0.005 | 0-0.005 | 0-0.005 | 0-0.005 |
| bronopol | 0-0.05 | 0-0.05 | 0-0.05 | 0-0.05 | 0-0.05 | 0-0.05 | 0-0.05 | 0-0.05 |
| Benzoic acid/Na- | 0-1 | 0-1 | 0-1 | 0-1 | 0-1 | 0-1 | 0-1 | 0-1 |
| benzoate | | | | | | | | |
| Benzylalcohol | 0-1 | 0-1 | 0-1 | 0-1 | 0-1 | 0-1 | 0-1 | 0-1 |
| Formic acid/Na- | 0-1 | 0-1 | 0-1 | 0-1 | 0-1 | 0-1 | 0-1 | 0-1 |
| Formate | | | | | | | | |
| Sorbic acid/K- | 0-1 | 0-1 | 0-1 | 0-1 | 0-1 | 0-1 | 0-1 | 0-1 |
| Sorbate | | | | | | | | |
| Perfumes/dyes/ | yes | yes | yes | yes | yes | yes | yes | yes |
| others | | | | | | | | |
| Hydrochloric acid or | 4-9 | 4-9 | 4-9 | 4-9 | 4-9 | 4-9 | 4-9 | 4-9 |
| sodium hydroxide to | | | | | | | | |
| reach pH | | | | | | | | |
| Deionized water | Up to | Up to | Up to | Up to | Up to | Up to | Up to | Up to |
| | 100% | 100% | 100% | 100% | 100% | 100% | 100% | 100% |

The formulations IX-b to XVI-b correspond to the same formulations as IX to XVI, but with Bacillus amyloliquefaciens (DSM 34304) instead of Bacillus methylotrophicus (DSM 34306).

The formulations IX-c to XVI-c correspond to the same formulations as IX to XVI, but with Bacillus amyloliquefaciens (DSM 34305) instead of Bacillus methylotrophicus (DSM 34306).

### Example 9—Detergent Formulations

In a preferred embodiment the bacterial strains of the invention are used in a laundry detergent.

Liquid laundry detergents according to the present invention are composed of:
0.05-20% of at least one (anionic, non-ionic or amphoteric) polymer
1-50% of surfactants
0.1-40% of builders, cobuilders and/or chelating agents
0.1-50% other adjuncts
0.001-5% of the bacterial strain(s) of the invention
water to add up 100%.

Preferred liquid laundry detergents according to the present invention are composed of (wt
0.2-6% of at least one (anionic, non-ionic or amphoteric) polymer
5-40% of anionic surfactants selected from C10-C15-LAS and C10-C18 alkyl ethersulfates containing 1-5 ethoxy-units
1.5-10% of non-ionic surfactants selected from C10-C18-alkyl ethoxylates containing 3-10 ethoxy-units
2-20% of soluble organic builders/co-builders selected from C10-C18 fatty acids, di- and tricarboxylic acids, hydroxy-di- and hydroxytricaboxylic acids and polycarboxylic acids
0.05-5% of an enzyme system containing at least one enzyme suitable for detergent use and preferably also an enzyme stabilizing system
0.5-20% of mono- or diols selected from ethanol, isopropanol, ethylenglycol, or propylenglyclol
0.1-20% other adjuncts
0.001-5% of the bacterial strain(s) of the invention
water to add up to 100%.

Solid laundry detergents (like e.g. powders, granules or tablets) according to the present invention are composed of:
0.05-20% of at least one (anionic, non-ionic or amphoteric) polymer
1-50% of surfactants
0.1-80% of builders, co-builders and/or chelating agents
0-50% fillers
0-40% bleach actives
0.1-30% other adjuncts and/or water
0.001-5% of the bacterial strain(s) of the invention
wherein the sum of the ingredients adds up 100%.

Preferred solid laundry detergents according to the present invention are composed of:
0.2-6% of at least one (anionic, non-ionic or amphoteric) polymer
5-30% of anionic surfactants selected from C10-C15-LAS, C10-C18 alkylsulfates and C10-C18 alkyl ethersulfates containing 1-5 ethoxy-units
1.5-7.5% of non-ionic surfactants selected from C10-C18-alkyl ethoxylates containing 3-10 ethoxy-units
5-50% of inorganic builders selected from sodium carbonate, sodiumbicarbonate, zeolites, soluble silicates, sodium sulfate
0.5-15% of cobuilders selected from C10-C18 fatty acids, di- and tricarboxylic acids, hydroxydi- and hydroxytricarboxylic acids and polycarboxylic acids
0.1-5% of an enzyme system containing at least one enzyme suitable for detergent use and preferably also an enzyme stabilizing system
0.5-20% of mono- or diols selected from ethanol, isopropanol, ethylenglycol, or propylenglyclol
0.1-20% other adjuncts
0.001-5% of the bacterial strain(s) of the invention
water to add up to 100%.

In a preferred embodiment the bacterial according to the present invention is used in a manual dish wash detergent.

Liquid manual dish wash detergents according to the present invention are composed of:
0.05-10% of at least one (anionic, non-ionic or amphoteric) polymer
1-50% of surfactants
0.1-50% of other adjuncts
0.001-5% of the bacterial strain(s) of the invention
water to add up 100%.

Preferred liquid manual dish wash detergents according to the present invention are composed of:
0.2-5% of at least one (anionic, non-ionic or amphoteric) polymer
5-40% of anionic surfactants selected from C10-C15-LAS, C10-C18 alkyl ethersulfates containing 1-5 ethoxy-units, and C10-C18 alkylsulfate
2-10% of Cocamidopropylbetaine
0-10% of Lauramine oxide
0-2% of a non-ionic surfactant, preferably a C10-Guerbet alcohol alkoxylate
0-5% of an enzyme, preferably Amylase, and preferably also an enzyme stabilizing system
0.5-20% of mono- or diols selected from ethanol, isopropanol, ethylenglycol, or propylenglyclol
0.1-20% other adjuncts
0.001-5% of the bacterial strain(s) of the invention
water to add up to 100%.

The following table shows general cleaning compositions of certain types, which correspond to typical compositions correlating with typical washing conditions as typically employed in various regions and countries of the world. The at least one inventive bacterial strain may be added to such formulation(s) in suitable amounts, such as 0.1 to 5% of the total weight.

General formula for laundry detergent compositions:

| | Ranges of Ingredient |
|---|---|
| Ingredient | in Liquid frame formulations |
| Linear alkyl benzene sulphonic acid | 0 to 30% |
| Coco fatty acid | 1 to 12% |
| Fatty alcohol ether sulphate | 0 to 25% |
| NaOH or mono or triethanol amine | Up to pH 7.5 to 9.0 |
| Alcohol ethoxylate | 3 to 10% |
| 1,2-Propylene glycol | 1 to 10% |
| Ethanol | 0 to 4% |
| Sodium citrate | 0 to 8% |
| water | Up to 100% |

1: A Bacillus strain having extracellular hydrolase activity and demonstrating reduced malodor after incubation for at least 5 days at a temperature of at least 25 °C in a lactose-free whole milk sample compared to a non-inoculated lactose-free whole milk sample.

2: The Bacillus strain according to claim 1, wherein the hydrolase activity is at least one selected from the group consisting of protease activity, amylase activity, lipase activity, mannanase activity, and cellulase activity.

3: The Bacillus strain according to claim 1, wherein the incubation is for at least 7 days at a temperature of at least 30 °C.

4: The Bacillus strain according to claim 1, wherein said strain has the ability form a spore.

5: The Bacillus strain according to claim 1, wherein said strain is selected from the group of Bacillus species consisting of Bacillus methylotrophicus, Bacillus subtilis and Bacillus amyloliquefaciens.

6: The Bacillus strain according to claim 1,
i) as deposited under accession number DSM 34306 or a mutant thereof;
ii) as deposited under accession number DSM 34307 or a mutant thereof;
iii) as deposited under accession number DSM 34304 or a mutant thereof; or
iv) as deposited under accession number DSM 34305 or a mutant thereof,
wherein:
the mutant in i), ii), iii) and iv) has the ability to form a spore, has extracellular hydrolase activity, and demonstrates reduced malodor after incubation for at least 7 days at a temperature of at least 30 °C in a lactose-free whole milk sample compared to a non-inoculated lactose-free whole milk sample; and
the hydrolase activity is at least one selected from the group consisting of protease activity, amylase activity, lipase activity, mannanase activity, and cellulase activity.

7: The Bacillus strain according to claim 6, wherein
i) the strain as deposited under accession number DSM 34306 comprises 16S rDNA having the sequence of SEQ ID NO:1;
ii) the strain as deposited under accession number DSM 34307 comprises 16S rDNA having the sequence of SEQ ID NO:2;
iii) the strain as deposited under accession number DSM 34304 comprises 16S rDNA having the sequence of SEQ ID NO:3; or
iv) the strain as deposited under accession number DSM 34305 comprises 16S rDNA having the sequence of SEQ ID NO:4.

8: The Bacillus strain according to claim 6, wherein
i) the mutant of the strain as deposited under accession number DSM 34306 comprises 16S rDNA having at least 98% sequence identity to SEQ ID NO:1;
ii) the mutant of the strain as deposited under accession number DSM 34307 comprises 16S rDNA having at least 98% sequence identity to SEQ ID NO:2;
iii) the mutant of the strain as deposited under accession number DSM 34304 comprises 16S rDNA having at least 98% sequence identity to SEQ ID NO:3; or
iv) the mutant of the strain as deposited under accession number DSM 34305 comprises 16S rDNA having at least 98% sequence identity to SEQ ID NO:4.

9: A composition comprising at least one Bacillus strain according to claim 1 and at least one additional component.

10: The composition according to claim 9, wherein the composition is a detergent composition, a sanitary or kitchen cleaner or an anti-malodor product.

11: The composition according to claim 9, wherein the composition comprises one or more surfactants and/or one or more builders and/or one or more preservative agents and/or one or more enzymes and/or one or more solvents.

12: A method to improve detergency of a detergent formulation comprising adding at least one Bacillus strain according to claim 1 to a detergent formulation.

13: A cleaning method comprising contacting at least one Bacillus strain according to claim 1 with an object that requires cleaning.

14: A method to reduce malodor comprising contacting at least one Bacillus strain according to claim 1 with an object that requires cleaning.

15: A method to control or reduce malodor-causing microorganisms comprising contacting at least one Bacillus strain according to claim 1 with an object or with a medium on/in which said microorganisms is present or grows.
